(19) United States
(12) Patent Application Publication
(10) Pub. No.: US 2014/0018566 A1
(43) Pub. Date: Jan. 16, 2014

(54) PALLADIUM(II)-CATALYZED SELECTIVE FLUORINATION OF BENZOIC ACIDS AND DERIVATIVES

(71) Applicant: JIN-QUAN YU, San Diego, CA (US)

(72) Inventor: JIN-QUAN YU, San Diego, CA (US)

(73) Assignee: The Scripps Research Institute, La Jolla, CA (US)

(21) Appl. No.: 13/546,616

(22) Filed: Jul. 11, 2012

(51) Int. Cl. C07C 231/14; C07C 253/30

(52) U.S. Cl. 558/415; 564/184

(57) ABSTRACT

A new method of ortho-fluorination for selectively fluorinating a benzoic acid or derivative compound where an aryl C—H bond is directly replaced by an aryl C—F bond is provided. The method comprises reacting a compound having the formula:

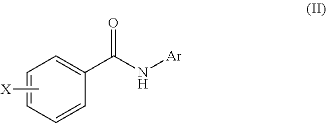

wherein X is at least one substitution group attached with a benzene ring and Ar is an aromatic substitution group comprising at least one fluorine atom,
   and a mixture comprising a palladium (II) catalyst and a fluorinating reagent to form at least one of the ortho-fluorinated compounds having the formulae

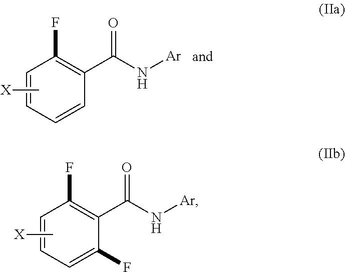

with good yield and selectivity.

### FIELD OF INVENTION

Embodiments of the invention are directed to methods for regioselectively replacing C—H bonds directly by C—F bonds. Compositions synthesized by these methods comprise fluorinated molecules.

### BACKGROUND

Fluorinated organic compound are valuable to pharmaceutical and agrochemical industries. For example, fluorinated arenes or aryl fluoride (ArF) moieties have long been recognized as privileged pharmacophores; not only are they isosteric to the parent, non-fluorinated arenes, but they exhibit improved lipophilicity as well as inertness to metabolic transformations (Shimizu, M.; Hiyama, T. Angew. Chem., Int. Ed. 2005, 44, 214; Tredwell, M.; Gouverneur, V. Org. Biomol. Chem. 2006, 4, 26; Mailer, K.; Faeh, C.; Diederich, F. Science 2007, 317, 1881). Therefore, development of new methods for the introduction of fluorines into arenes is a significant task.

Pd⁰-catalyzed displacement of aryl bromides and aryl triflates by the nucleophilic fluoride anion has also been demonstrated (Watson, D. A.; Su, M.; Teverovskiy, G.; Zhang, Y.; Garcia-Fortanet, J.; Kinzel. T.; Buchwald, S. L. Science 2009, 325, 1661). However, the development of methods to fluorinate directly unactivated aryl C—H bonds has met with limited success. Only two examples of directed ortho-C—H fluorination have been reported (Hull, K. L; Anani, W. Q.; Sanford, M. S. J. Am. Chem. Soc. 2006, 128, 7134; Wang, X.; Mei, T.-S.; Yu, J.-Q. J. Am. Chem. Soc. 2009, 131, 75200). In both of these cases, formation of a mixture of inseparable mono- and di-fluorinated arenes is often problematic for practical applications.

### SUMMARY

This Summary is provided to present a summary of the invention to briefly indicate the nature and substance of the invention. It is submitted with the understanding that it will not be used to interpret or limit the scope or meaning of the claims.

The present disclosure provides a new method of ortho-fluorination for selectively fluorinating a benzoic acid or derivative compound where an aryl C—H bond is directly replaced by an aryl C—F bond.

In accordance with some embodiments, the method comprises three steps while the first and third steps described below may be optional in some embodiments. The first step comprises reacting a benzoic acid or derivative compound having the formula (I) to form a benzamide compound having the formula (II), according to Scheme A:

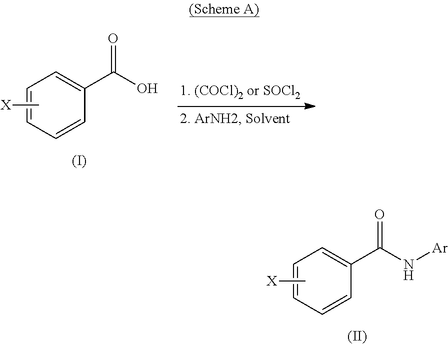

wherein X is at least one substitution group attached with a benzene ring and Ar is an aromatic substitution group comprising at least one fluorine atom. X can be any substitution group. Examples of X are described infra.

The second step includes reacting a compound having the formula (II), and a mixture comprising a palladium (II) catalyst, a fluorinating reagent, and optionally an additive and a solvent, to form at least one of the ortho-fluorinated compounds having the formulae (IIa) and (IIb), with good yield and selectivity, according to Scheme B.

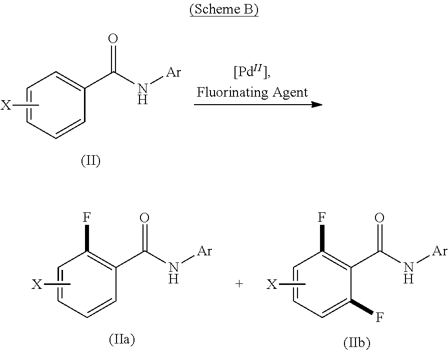

The third step involves transforming the at least one of the ortho-fluorinated compounds having the formula (IIa) and (IIb) into an ortho-fluorinated benzoic acid or derivative compound having the formula (IIIa) or (IIIb), according to Scheme C or D:

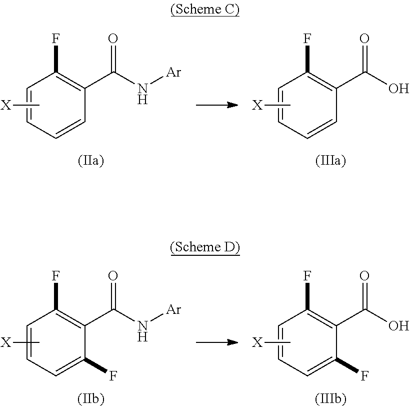

In general, this disclosure provides a method for selectively fluorinating a benzoic acid or derivative compound, which comprises reacting a compound having the formula (II), and a mixture comprising a palladium (II) catalyst, a fluorinating reagent, and optionally an additive and a solvent, to form at least one of ortho-fluorinated compounds having the formulae (IIa) and (IIb) with good yield and selectivity, in accordance with some embodiments. This method is illustrated in Scheme B. The yield of either the monofluorinated compound (IIa) or the difluorinated compound (IIb) is higher than 50%, preferably at least 80% while the yield of the other fluorinated compound (IIb or IIa) is lower than 10%, preferably lower than 5%.

Other aspects are described infra.

### DETAILED DESCRIPTION

Several aspects of the invention are described below with reference to example applications for illustration. It should be understood that numerous specific details, relationships, and methods are set forth to provide a full understanding of the invention. One having ordinary skill in the relevant art, however, will readily recognize that the invention can be practiced without one or more of the specific details or with other methods. The present invention is not limited by the illustrated ordering of acts or events, as some acts may occur in different orders and/or concurrently with other acts or events. Furthermore, not all illustrated acts or events are required to implement a methodology in accordance with the present invention.

### DEFINITIONS

The terminology used herein is for the purpose of describing particular embodiments only and is not intended to be limiting of the invention. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

As used herein, the term “alkyl” refers to an optionally substituted, saturated, straight or branched hydrocarbon having at least one carbon atoms, such as about 1 to about 30 carbon atoms (and all combinations and subcombinations of ranges and specific numbers of carbon atoms therein), preferably with from about 1 to about 8 carbon atoms, more preferably with from about 1 to about 6 carbon atoms, even more preferably with from about 1 to about 4 carbon atoms, still more preferably with from about 1 to about 3 carbon atoms, with 1 carbon being especially preferred in some embodiments. For example, the term “C₁₋₆ alkyl” means a straight or branched alkyl containing at least 1 and at most 6 carbon atoms. In some embodiments, the alkyl is optionally substituted. For example, 1 or more of the hydrogen atoms on the alkyl group, preferably from 1 to about 6, more preferably about 1 to about 3 of hydrogen atoms on the alkyl group are substituted with a F, Cl, Br, NH₂, NO₂, N₃, CN, COOH, OH, etc. Alkyl groups include, but are not limited to, methyl, ethyl, n-propyl, isopropyl, n-butyl, isobutyl, sec-butyl, t-butyl, n-pentyl, isopentyl, neopentyl, n-hexyl, isohexyl, 3-methylpentyl, 2,2-dimethylbutyl, and 2,3-dimethylbutyl.

As used herein, the term “cycloalkyl” refers to an optionally substituted, mono-, di-, tri-, or other multicyclic alicyclic ring system having at least three carbon atoms, such as from about 3 to about 20 carbon atoms (and all combinations and subcombinations of ranges and specific numbers of carbon atoms therein). In some preferred embodiments, the cycloalkyl groups have from about 3 to about 10 carbon atoms, more preferably from about 3 to about 8 carbon atoms, with from about 3 to about 6 carbon atoms being preferred. For example, the term “C₃₋₆ cycloalkyl” means a mono- or bicyclic saturated ring structure containing at least 3 and at most 6 carbon atoms. Multi-ring structures may be bridged or fused ring structures, wherein the additional groups fused or bridged to the cycloalkyl ring may include optionally substituted cycloalkyl, aryl, heterocycloalkyl, or heteroaryl rings. Exemplary cycloalkyl groups include, but are not limited to, cyclopropyl, cyclobutyl, cyclopentyl, cyclohexyl, cyclooctyl, adamantyl, 2-[4-isopropyl-1-methyl-7-oxa-bicyclo[2.2.1]-heptanyl], and 2-[1,2,3,4-tetrahydro-naphthalenyl].

As used herein, the term “alkylcycloalkyl” refers to an optionally substituted ring system comprising a cycloalkyl group substituted with one or more alkyl substituents, wherein cycloalkyl and alkyl are each as previously defined. Exemplary alkylcycloalkyl groups include 2-methylcyclohexyl, 3,3-dimethylcyclopentyl, trans-2,3-dimethylcyclooctyl, and 4-methyldecahydronaphthalenyl.

As used herein, the term “alkenyl” as a group or a part of a group refers to an optionally substituted straight or branched hydrocarbon chain containing the specified number of carbon atoms and containing at least one double bond. For example, the term “C₂₋₆ alkenyl” means a straight or branched alkenyl containing at least 2 and at most 6 carbon atoms and containing at least one double bond. Multiple double bonds may be adjacent (═C═), conjugated (═C—C═), or are non-adjacent and non-conjugated. In particular, multiple double bonds are conjugated, or are non-adjacent and non-conjugated. It will be appreciated that in groups of the form —O—C₂₋₆ alkenyl, the double bond is preferably not adjacent to the oxygen. Preferably, the alkenyl groups of the invention may be optionally substituted and have from about 2 to about 10 carbon atoms (and all combinations and subcombinations of ranges and specific numbers of carbon atoms therein), more preferably 2 to 6 carbon atoms. Examples of “alkenyl” as used herein include, but are not limited to, ethenyl, 2-propenyl, 3-butenyl, 2-butenyl, 2-pentenyl, 3-pentenyl, 3-methyl-2-butenyl, 3-methylbut-2-enyl, 3-hexenyl and 1,1-dimethylbut-2-enyl.

The term “alkynyl” as used herein as a group or a part of a group refers to an optionally substituted straight or branched hydrocarbon chain containing the specified number of carbon atoms and containing at least one triple bond. For example, the term “C₂₋₆ alkynyl” means a straight or branched alkynyl containing at least 2, and at most 6, carbon atoms and containing at least one triple bond. Multiple triple bonds may be conjugated or non-conjugated. In particular, multiple triple bonds are non-conjugated. It will be appreciated that in groups of the form —O—C₂₋₆ alkynyl, the triple bond is preferably not adjacent to the oxygen. Preferably, the alkynyl groups of the invention may be optionally substituted and have from about 2 to about 10 (and all combinations and subcombinations of ranges and specific numbers of carbon atoms therein) carbon atoms, preferably 2 to 6 carbon atoms. Examples of “alkynyl” as used herein include, but are not limited to, ethynyl, 2-propynyl, 3-butynyl, 2-butynyl, 2-pentynyl, 3-pentynyl, 3-methyl-2-butynyl, 3-methylbut-2-ynyl, 3-hexynyl and 1,1-dimethylbut-2-ynyl.

As used herein, the term “aryl” refers to an optionally substituted, mono-, di-, tri-, or other multicyclic aromatic ring system having at least 6 carbon atoms, for example, from about 6 to about 50 carbon atoms (and all combinations and subcombinations of ranges and specific numbers of carbon atoms therein), preferably with from about 6 to about 14 carbons, with about 6 to 10 carbon atoms being preferred. Non-limiting examples include, for example, phenyl, naphthyl, anthracenyl, and phenanthrenyl. Aryl may optionally be further fused to an aliphatic or aryl group or can be substituted with one or more substituents such as halogen (fluorine, chlorine and/or bromine), hydroxy, alkyl, alkoxy or aryloxy, amido, nitro, alkylenedioxy, alkylthio or arylthio, alkylsulfonyl, cyano, or primary, secondary or tertiary amino.

As used herein, the term “alkoxy” refers to an optionally substituted straight or branched chain alkyl —O— group wherein alkyl is as previously defined. For example, C₁₋₆ alkoxy means a straight or branched alkoxy containing at least 1, and at most 6, carbon atoms. Examples of “alkoxy” as used herein include, but are not limited to, methoxy, ethoxy, propoxy, prop-2-oxy, butoxy, but-2-oxy, 2-methylprop-1-oxy, 2-methylprop-2-oxy, pentoxy and hexyloxy. A C₁₋₄ alkoxy group is preferred, for example methoxy, ethoxy, propoxy, prop-2-oxy, butoxy, but-2-oxy or 2-methylprop-2-oxy. In some preferred embodiments, the alkyl moieties of the alkoxy groups have from about 1 to about 4 carbon atoms. As used herein, the term “aryloxy” refers to an optionally substituted aryl-O-group wherein aryl is as previously defined. Exemplary aryloxy groups include, but are not limited to, phenoxy (phenyl-O—) and naphthoxy (naphthyl-O).

As used herein, the term “heteroaryl” refers to an optionally substituted aryl ring system wherein, in at least one of the rings, one or more of the carbon atom ring members is independently replaced by a heteroatom group selected from the group consisting of S, O, N, and NH, or NR wherein aryl is as previously defined and R is an optional substitutent as defined herein. Heteroaryl groups having a total of from about 5 to about 14 carbon atom ring members and heteroatom ring members (and all combinations and subcombinations of ranges and specific numbers of carbon and heteroatom ring members) are preferred. Heteroaryl groups having a total of from about 5 to about 10 carbon atom ring members and heteroatom ring members (and all combinations and subcombinations of ranges and specific numbers of carbon and heteroatom ring members) are more preferred. Exemplary heteroaryl groups include, but are not limited to, pyrryl, furyl, pyridyl, pyridine-N-oxide, 1,2,4-thiadiazolyl, pyrimidyl, thienyl, isothiazolyl, imidazolyl, tetrazolyl, pyrazinyl, pyrimidyl, quinolyl, isoquinolyl, thiophenyl, benzothienyl, isobenzofuryl, pyrazolyl, indolyl, purinyl, carbazolyl, benzimidazolyl, and isoxazolyl. Heteroaryl may be attached to the rest of the molecule via a carbon or a heteroatom.

As used herein, the term “heteroarylalkyl” refers to an optionally substituted ring system comprising an alkyl radical bearing a heteroaryl substituent, each as defined above, having at least 6 carbon atoms, for example, from about 6 to about 50 carbon atoms (and all combinations and subcombinations of ranges and specific numbers of carbon atoms therein), with from about 6 to about 25 carbon atoms being preferred. Non-limiting examples include 2-(1H-pyrrol-3-yl)ethyl, 3-pyridylmethyl, 5-(2H-tetrazolyl)methyl, and 3-(pyrimidin-2-yl)-2-methylcyclopentanyl.

As used herein, the term “heterocycloalkyl,” “heterocyclic ring” and “heterocyclyl” each refer to an optionally substituted ring system composed of a cycloalkyl radical wherein, in at least one of the rings, one or more of the carbon atom ring members is independently replaced by a heteroatom group selected from the group consisting of O, S, N, and NH, or NR wherein cycloalkyl is as previously defined and R is an optional substituent as defined herein. Heterocycloalkyl ring systems having a total of from about 3 to about 14 carbon atom ring members and heteroatom ring members (and all combinations and subcombinations of ranges and specific numbers of carbon and heteroatom ring members) are preferred, more preferably from about 3 to about 10 ring atom members. In other preferred embodiments, the heterocyclic groups may be fused to one or more aromatic rings. In certain preferred embodiments, heterocycloalkyl moieties are attached via a ring carbon atom to the rest of the molecule. Exemplary heterocycloalkyl groups include, but are not limited to, azepanyl, tetrahydrofuranyl, hexahydropyrimidinyl, tetrahydrothienyl, piperidinyl, pyrrolidinyl, isoxazolidinyl, isothiazolidinyl, pyrazolidinyl, oxazolidinyl, thiazolidinyl, piperazinyl, 2-oxo-morpholinyl, morpholinyl, 2-oxo-piperidinyl, piperadinyl, decahydroquinolyl, octahydrochromenyl, octahydro-cyclopentapyranyl, 1,2,3,4,-tetrahydroquinolyl, 1,2,3,4-tetrahydroquinazolinyl, octahydro-[2]pyridinyl, decahydro-cyclooctafuranyl, 1,2,3,4-tetrahydroisoquinolyl, 2-oxo-imidazolidinyl, and imidazolidinyl. In some embodiments, two moieties attached to a heteroatom may be taken together to form a heterocycloalkyl ring. In certain of these embodiments, 1 or 2 of the heterocycloalkyl ring carbon atoms may be replaced by other moieties which contain either one (—O—, —S—, —N(R⁹)—) or two) (—N(R¹⁰—C(═O)—, or —C(═O)—N(R¹⁰—) ring replacement atoms. When a moiety containing one ring replacement atom replaces a ring carbon atom, the resultant ring, after replacement of a ring atom by the moiety, will contain the same number of ring atoms as the ring before ring atom replacement. When a moiety containing two ring replacement atoms replaces a ring carbon atom, the resultant ring after replacement will contain one more ring atom than the ring prior to replacement by the moiety. For example, when a piperidine ring has one of its ring carbon atoms replaced by —N(R¹⁰—C(═O)—, the resultant ring is a 7-membered ring containing 2 ring nitrogen atoms and the carbon of a carbonyl group in addition to 4 other carbon ring atoms (CH₂ groups) from the original piperidine ring. In general, the ring system may be saturated or may be partially unsaturated, i.e., the ring system may contain one or more non-aromatic C—C or C—N double bonds.

The term “optionally substituted” means that group in question may be unsubstituted or it may be substituted one or several times, such as 1 to 3 times or 1 to 5 times. For example, an alkyl group that is “optionally substituted” with 1 to 5 chloro atoms, may be unsubstituted, or it may contain 1, 2, 3, 4, or 5 chlorine atoms.

Typically, substituted chemical moieties include one or more substituents that replace hydrogen. Exemplary substituents include, for example, halo (e.g., F, Cl, Br, I), alkyl, cycloalkyl, alkylcycloalkyl, alkenyl, alkynyl, haloalkyl including trifluoroalkyl, aralkyl, aryl, heteroaryl, heteroarylalkyl, spiroalkyl, heterocyclyl, heterocycloalkyl, hydroxyl (—OH), alkoxyl, aryloxyl, aralkoxyl, nitro (—NO₂), cyano (—CN), amino (—NH₂), N-substituted amino (—NHR″), N,N-disubstituted amino (—N(R″)R″), carboxyl (—COOH), —C(═O)R″, —OR″, —C(═O)OR″, —C(═O)NHSO₂R″, —NHC(═O)R″, aminocarbonyl (—C(═O)NH₂), N-substituted aminocarbonyl (—C(═O)NHR″), N,N-disubstituted aminocarbonyl (—C(═O)N(R″)R″), thiolato (SR″), sulfonic acid and its esters (—SO₃R″), phosphonic acid and its mono-ester (—P(═O)(OR″)(OH) and di-esters (—P(═O)(OR″)(OR″), —S(═O)₂R″, —S(═O)₂NH₂, —S(═O)₂NHR″, —S(═O)₂NR″R″, —SO₂NHC(═O)R″, —NHS(═O)₂R″, —NR″S(═O)₂R″, —CF₃, —CF₂CF₃, —NHC(═O)NHR″, —NHC(═O)NR″R″, —NR″C(═O)NHR″, —NR″C(═O)NR″R″, —NR″C(═O)R″ and the like. In relation to the aforementioned substituents, each moiety “R” can be, independently, any of H, alkyl, cycloalkyl, alkenyl, aryl, aralkyl, heteroaryl, or heterocycloalkyl, or when (R″(R″)) is attached to a nitrogen atom, R″ and R″ can be taken together with the nitrogen atom to which they are attached to form a 4- to 8-membered nitrogen heterocycle, wherein the heterocycloalkyl ring is optionally interrupted by one or more additional —O—, —S—, —SO, —SO₂—, —NH—, —N(alkyl)-, or —N(aryl)-groups, for example. In certain embodiments, chemical moieties are substituted by at least one optional substituent, such as those provided hereinabove. In the present invention, when chemical moieties are substituted with optional substituents, the optional substituents are not further substituted unless otherwise stated. For example; when R₁ is an alkyl moiety, it is optionally substituted, based on the definition of “alkyl” as set forth herein. In some embodiments, when is alkyl substituted with optional aryl, the optional aryl substituent is not further substituted.

When any variable occurs more than one time in any constituent or formula for a compound, its definition at each occurrence is independent of its definition at every other occurrence. Thus, for example, if the R₅ group is shown to be substituted with 0-2 substituents, then said group may optionally be substituted with up to two substituents and each substituents is selected independently from the definition of optionally substituted defined above. Also, combinations of substituents and/or variables are permissible only if such combinations result in stable compounds.

When a bond to a substituent is shown to cross a bond connecting two atoms in a ring, then such substituent may be bonded to any atom on the ring having an attached hydrogen atom. When a substituent is listed without indicating the atom via which such substituent is bonded to the rest of the compound of a given formula, then such substituent may be bonded via any atom in such substituent. Combinations of substituents and/or variables are permissible only if such combinations result in stable compounds.

The term “chiral” refers to molecules which have the property of non-superimposability of the mirror image partner, while the term “achiral” refers to molecules which are superimposable on their mirror image partner.

The term “diastereomers” refers to stereoisomers with two or more centers of dissymmetry and whose molecules are not mirror images of one another. The term “enantiomers” refers to two stereoisomers of a compound which are non-superimposable mirror images of one another. An equimolar mixture of two enantiomers is called a “racemic mixture” or a “racemate.” The term “isomers” or “stereoisomers” refers to compounds which have identical chemical constitution, but differ with regard to the arrangement of the atoms or groups in space.

Furthermore the indication of configuration across a carbon-carbon double bond can be “Z” referring to what is often referred to as a “cis” (same side) conformation whereas “E” refers to what is often referred to as a “trans” (opposite side) conformation. Regardless, both configurations, cis/trans and/or Z/E are contemplated for the compounds for use in the present invention. With respect to the nomenclature of a chiral center, the terms “d” and “I”, “R” and “S”, configuration are as defined by the IUPAC Recommendations. As to the use of the terms, diastereomer, racemate, epimer and enantiomer, these will be used in their normal context to describe the stereochemistry of preparations.

Unless specifically stated herein, the compounds as described herein may contain any stereoisomer, racemate, or a mixture thereof.

Natural amino acids represented by the compounds utilized in the present invention are in the “L” configuration, unless otherwise designated. Unnatural or synthetic amino acids represented by the compounds utilized in the present invention may be in either the “D” or “L” configurations.

Another aspect is a radiolabeled compound of any of the formulae delineated herein. Such compounds have one or more radioactive atoms (e.g., ³H, ²H, ¹⁴C, ¹³C, ³⁵S, ³²P, ¹²⁵I, ¹³¹I) introduced into the compound. Such compounds are useful for drug metabolism studies and diagnostics, as well as therapeutic applications.

“Substituted” refers to a group in which one or more hydrogen atoms are each independently replaced with the same or different substituent(s).

Terminology related to “protecting”, “deprotecting” and “protected” functionalities occurs throughout this application. Such terminology is well understood by persons of skill in the art and is used in the context of processes, which involve sequential treatment with a series of reagents. In that context, a protecting group refers to a group that is used to mask a functionality during a process step in which it would otherwise react, but in which reaction is undesirable. The protecting group prevents reaction at that step, but may be subsequently removed to expose the original functionality. The removal or “deprotection” occurs after the completion of the reaction or reactions in which the functionality would interfere. Protection and deprotection of functional groups may be performed by methods known in the art (see, for example, Green and Wuts Protective Groups in Organic Synthesis. John Wiley and Sons, New York, 1999.). In addition to the protecting groups as described elsewhere, hydroxyl or amino groups may be protected with any hydroxyl or amino protecting group. The amino protecting groups may be removed by conventional techniques. For example, acyl groups, such as alkanoyl, alkoxycarbonyl and aroyl groups, may be removed by solvolysis, e.g., by hydrolysis under acidic or basic conditions. Arylmethoxycarbonyl groups (e.g., benzyloxycarbonyl) may be cleaved by hydrogenolysis in the presence of a catalyst such as palladium-on-charcoal.

Fluorinated Compounds

A practical protocol for Pd-catalytic C—H fluorination using electrophilic fluorine is lacking. The inventors have determined that there are two major obstacles for developing reactions of this type. First, directed palladation of C—H bonds is significantly inhibited by the fluorinating reagents. It has been proposed that the supporting pyridine ligand of the fluorinating reagents either competes with the directing group for the binding site at the Pd^{II} center, or the electrophilic fluorine could complex with the Lewis basic directing group and weaken its ability to bind to Pd^{II}. Second, the use of Pd(OAc)₂, a broadly useful catalyst for C—H functionalization, would lead to the formation of a [Pd^{IV}(OAc)(F)] species following C—H activation and oxidation with r, from which the C—OAc reductive elimination outcompetes the relatively slow C—F reductive elimination.

To address the first problem, a strongly coordinating pyridine directing group has been tried to ensure cyclometallation with 2-phenylpyridines (Hull, K. L.; Anani, W. Q.; Sanford, M. S. J. Am. Chem. Soc. 2006, 128, 7134). An X-type anionic triflamide directing group has been devised to avoid direct competition with the pyridine moieties contained in fluorinating reagents (Scheme E). In addition, to mitigate the second problem, Pd(OTf)₂ is used as the catalyst in Scheme E to ensure selective C—F reductive elimination instead of C—OAc reductive elimination. This method is described in the U.S. Patent Application Publication No. 2012/0059179 in the name of Jin-Quan Yu assigned to the Scripps Research Institute.

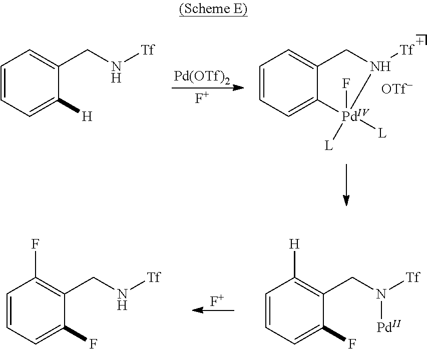

where Tf is triflate (trifluoro-methanesulfonyl).

Although triflamides can be converted to other functional groups to meet synthetic needs, the necessity for the presence of ortho- or meta-substitution to prevent di-fluorination is a significant limitation of that chemistry. The formation of the di-fluorination product in Scheme E is probably due to the slow displacement of the X-type mono-fluorinated product attached to Pd^{II} center by the substrate. The use of a strongly coordinating L-type directing group such as pyridine caused similar problems. The inventors have envisioned that the use of a weak coordinating L-type acidic amide could allow for rapid displacement of the mono-fluorinated product by the substrate, thereby affording mono-selectivity.

Embodiments are directed to methods for ortho-fluorination of benzoic acids and derivatives using palladium (II) catalysis. The methods are used to make fluorinated benzoic acids or derivatives with good yield and selectivity in either monofluorinated or difluorinated products.

In general, the method may comprise three steps while the first and third steps described below may be optional in some embodiments. The first step comprises reacting a benzoic acid or derivative compound having the formula (I) to form a benzamide compound having the formula (II), according to Scheme A:

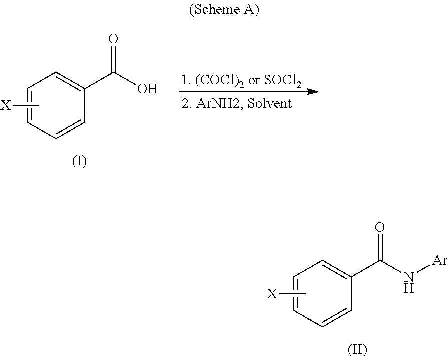

wherein X is at least one substitution group attached with a benzene ring and Ar is an aromatic substitution group comprising at least one fluorine atom, as described later.

The second step includes reacting a compound having the formula (II), and a mixture comprising a palladium (II) catalyst, a fluorinating reagent, and optionally an additive and a solvent, to form at least one of the Ortho-fluorinated compounds having the formulae (IIa) and (IIb), with good yield and selectivity, according to Scheme B.

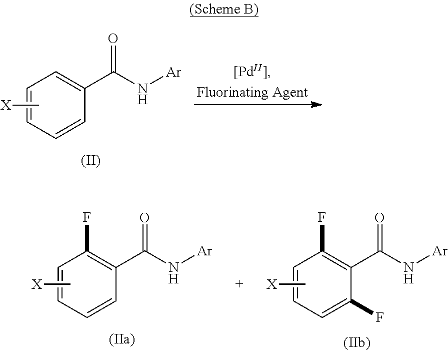

The third step involves transforming the at least one of the ortho-fluorinated compounds having the formula (IIa) and (IIb) into an ortho-fluorinated benzoic acid or derivative compound having the formula (IIIa) or (IIIb), according to Scheme C or D:

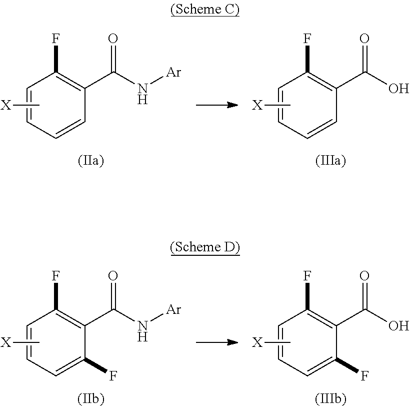

Details of the first step and third steps are described more in details in the examples, and some procedures generally known to those of ordinary skill in the art can be used.

In more general, in accordance with some embodiments, this disclosure also provides a method for selectively fluorinating a benzoic acid or derivative compound, which comprises reacting a compound having the formula (II), and a mixture comprising a palladium (II) catalyst, a fluorinating reagent, and optionally an additive and a solvent, to form at least one of ortho-fluorinated compounds having the formulae (IIa) and (IIb) with good yield and selectivity. This method is shown in Scheme B. The yield of either the monofluorinated compound (IIa) or the difluorinated compound (IIb) is higher than 50%, preferably at least 80% while the yield of the other fluorinated compound (IIb or IIa) is lower than 10%, preferably lower than 5%.

Other embodiments of the invention describe a novel method for regioselectivity replacing C—H bonds directly by fluorines. The directing groups used to control the regioselectivity can be subsequently converted to a wide range of synthetically desirable functional groups, thereby make this method extremely versatile for make a variety of fluorinated molecules of pharmaceutical interests.

X can be any substitution group. For example, X is at least one substitution group independently selected from the group of radicals consisting of H, halo, C₁₋₂₀ alkyl, C₂₋₂₀ alkenyl, C₂₋₂₀ alkynyl, C₁₋₂₀ alkoxyl, C₆₋₂₀ aryl, C₇₋₂₀ alkylaryl, C₄₋₂₀ heterocycle, C₄₋₂₀ heteroaryl, C₄₋₂₀ alkyl heterocycle, C₇₋₂₀ alkyl heteroaryl, C₆₋₂₀ aryloxy, —OH, —CO, —COOH, —NO₂, —CN, —NH₂, —N₃, —CF₃, —OCF₃, —NR³R³′, —N(O)R³, —SH, —SR³, —SOR³, —SO₂R³, —C(O)R³, —CO₂R³, —C(O)NR₃R³′, and —OC(O)R³; wherein the alkyl, alkenyl, alkynyl, alkoxy, aryl, heterocycle, heteroaryl, or aryloxy may be substituted or unsubstituted and wherein in the alkyl portion one or more —CH₂—, CH₂CH₂—, —(CH₂)ₙ— groups are each optionally replaced by —O— or —NH—; wherein n is an integer equal to or greater than 1; R³ and R³′ are each independently selected from the group of radicals consisting of H, C₁₋₂₀ alkyl, C₂₋₂₀ alkenyl, C₂₋₂₀ alkynyl, C₆₋₂₀ aryl; C₇₋₂₀ alkylaryl, C₄₋₂₀ heterocycle, C₄₋₂₀ heteroaryl, C₄₋₂₀ alkylheterocycle, and C₇₋₂₀ alkylheteroaryl; wherein the alkyl, alkenyl, alkynyl, aryl, heterocycle, or heteroaryl, be substituted or unsubstituted and wherein in the alkyl portion one or more —CH₂—, CH₂CH₂—, —(CH₂)ₙ— groups are each optionally replaced by —O— or —NH—; n is an integer equal to or greater than 1.

In some embodiments, X is one substitution group independently selected from the group of radicals consisting of H, halo, C₁₋₆ alkyl, C₂₋₆ alkenyl, C₂₋₆ alkynyl, C₁₋₆ alkoxyl, C₆₋₂₀ aryl; C₇₋₂₀ alkylaryl, C₄₋₂₀ heterocycle, C₄₋₂₀ heteroaryl, C₄₋₂₀ alkylheterocycle, and C₇₋₂₀ alkylheteroaryl; —OH, —CO, —COOH, —NO₂, —CN, —NH₂, —N₃, —CF₃, —OCF₃, —NR³R³′, —N(O)R³, —SH, —SR³, —SOR³, —SO₂R³, —C(O)R³, —CO₂R³, —C(O)NR³R³′, and —OC(O)R³; wherein the alkyl, alkenyl, alkynyl, alkoxy, aryl, heterocycle, heteroaryl, or aryloxy may be substituted or unsubstituted and wherein in the alkyl portion one or more: —CH₂—, CH₂CH₂—, —(CH₂)ₙ— groups are each optionally replaced by —O— or —NH—; wherein n is an integer equal to or greater than 1.

In some embodiments, at least two of the at least one X group are joined together to form a bicyclic or tricyclic ring structure with the benzene ring to which at least two of the at least one X group are attached, the bicyclic or tricyclic ring structure selected from the group consisting of alkyl, aryl, heterocycle and heteroaryl ring structures.

Ar is an aromatic substitution group comprising at least one fluorine atom. In some embodiments, Ar is an aryl group comprising at least two fluorine substitution groups. Preferred examples of Ar include but are not limited to a 2,3,5,6-tetrafluoro-4-(trifluoromethyl) phenyl group or a 2,3,5,6-tetrafluoro-4-cyano phenyl group.

The present invention involves palladium-catalyzed reaction. In principle, any known palladium(H) catalyst may be used. In one embodiment, the catalyst is 5% or more palladium(II). However, other catalysts having a faster reaction rate are preferred. For example, in some preferred embodiments, the palladium (II) catalyst is Pd (OTs)₂(CH₃CN)₄, Pd(OTf)₂(CH₃CN)₄, Pd(OTf)₂(H₂O)₄, Pd(NTf₂)₂, Pd(OTf)₂ or Pd(TFA)₂, wherein Ts is toluenesulfonyl, Tf is trifluoromethanesulfonyl and TFA is trifluoroacetate. More preferably, the palladium (II) catalyst is Pd(OTf)2(CH3CN)4.

The catalyst is generally employed in catalytically effective amounts. See, for example the Examples section which follows. For example, 5-20 mol % of the palladium (II) catalyst can be used.

The fluorinating agent is a compound having the formula:

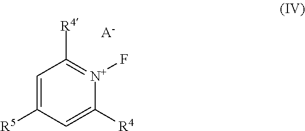

wherein A- is a counter ion; and R⁴, R⁴′, and R⁵ are each independently hydrogen, halogen, C₁₋₁₂ alkyl, or C₂₋₁₂ alkenyl, wherein the alkyl or alkenyl may be substituted with one or more halogen. In some embodiments, A- is OTf⁻, BF4⁻, or PF6⁻.

In some embodiments, the fluorinating agent is a compound having the formula (IV), R⁴ and R⁴′ are each independently halogen, C₁₋₆ alkyl; R⁵ is H or a C₁₋₆ alkyl; and A- is OTf⁻, BF4⁻, or PF6⁻. In some embodiments, the fluorinating reagent is N-fluoro-2-4-6-trimethylpyridinium triflate. In some embodiments, 1-5 equivalents of the fluorinating reagent is used.

In general, any fluorinating reagent may be used. In one embodiment, the fluorinating agent is an electrophilic fluorinating agent.

The mixture comprising a palladium (II) catalyst and a fluorinating reagent further comprises an additive and a solvent, in accordance with some embodiments.

An additive can be a promoter. In some embodiments, an additive is a compound having the formula:

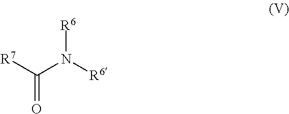

wherein R⁶ and R⁶′ are each H or a C₁₋₆ alkyl, wherein at least one of R⁶ and R⁶′ is not H, R⁷ is H, or a C₁₋₆ alkyl, or wherein either R⁶ and R⁶′ together with the nitrogen to which they are attached or R⁶ and R⁷ together with the nitrogen and carbonyl to which they are attached form a 5- or 6-membered ring which may be unsubstituted or substituted with one or more C₁₋₆ alkyl groups.

In some embodiments, the additive is N-methyl-2-pyrrolidone (NMP). It has been found that this compound is particularly useful to increase both the rate of the reaction and the yield for monofluorinated or difluorinated products.

In one embodiment, 0.1-5 equivalents of the additive is used. In some embodiments, 0.3-0.7 equivalents of the additive is used. In some embodiments, the identity and amount of the additive is adjusted to obtain the optimal yield of the preferred product.

The fluorination reaction in this disclosure is generally carried out in an aprotic solvent, which may be apolar or polar. Protic solvents solvate anions (negatively charged solutes) strongly via hydrogen bonding. Water is a protic solvent. Aprotic solvents such as acetone or dichloromethane tend to have large dipole moments (separation of partial positive and partial negative charges within the same molecule) and solvate positively charged species via their negative dipole. In chemical reactions the use of polar protic solvents favors the SN₁ reaction mechanism, while polar aprotic solvents favor the SN₂ reaction mechanism. Any aprotic solvent can be used.

In some embodiments, the solvent is trifluoromethyl benzene (PhCF3), ethyl acetate (EtOAc) or CH₃CN. In some embodiments, the solvent is preferably CH3CN for high selectivity in monofluorination of benzoic acids and derivatives. In some embodiments, the solvent is preferably PhCF₃ for high selectivity in difluorination of benzoic acids and derivatives.

Reacting the benzamide compound having the formula (II) to form at least one of the ortho-fluorinated compounds having the formulae (IIa) and (IIb) is performed at a temperature under a heating condition. In some embodiments, any of the reactions described herein can be carried out in a wide range of temperatures. For example, a reaction may be carried out at any temperature of from room temperature (about 23° C.) to 180 degree C., or up to the boiling temperature of the solvent. In some embodiments, the temperature is in the range of 80-120° C. In combination with selection of additive, solvent and fluorinating agents, temperature can be adjusted to adjust the selectivity in either monofluorinated or difluorinated products, as illustrated in Examples.

In some embodiments, the catalytic turnover and the reaction time takes between about 5 minutes to 12 hours. In some embodiments, the reaction takes 5 minutes to 4 hours. In some embodiments, the reaction time is about one to two hours.

In some embodiments of the disclosed fluorination method for monofluorinated products with good yield and selectivity, a combination of the following factors is used. In such combination, Ar is a 2,3,5,6-tetrafluoro-4-(trifluoromethyl) phenyl group; the palladium (II) catalyst is Pd(OTf)₂(CH₃CN)₄; the fluorinating reagent is N-fluoro-2-4-6-trimethylpyridinium triflate; the additive is NMP; and the solvent is CH₃CN.

In some embodiments, the yield of monofluorinated product having the formula (IIa) is at least 50%, and the yield of difluorinated product having the formula (IIb) is less than 10%. More preferably, the yield of monofluorinated product having the formula (IIa) is at least 80%, and the yield of difluorinated product having the formula (IIb) is less than 5%.

In some other embodiments, the yield of difluorinated product having the formula (IIb) is at least 50%, and the yield of monofluorinated product having the formula (IIa) is less than 10%. More preferably, wherein the yield of difluorinated product having the formula (IIb) is at least 80%, and the yield of monofluorinated product having the formula (IIa) is less than 5%.

In some embodiments of the fluorination method, provides for difluorinated products with good yield and selectivity, a combination of the following factors is used. In such combination, Ar is a 2,3,5,6-tetrafluoro-4-(trifluoromethyl)phenyl group; the palladium (II) catalyst is Pd(OTf)₂(CH₃CN)₄; the fluorinating reagent is N-fluoro-2-4-6-trimethylpyridinium triflate; the additive is NMP; and the solvent is PhCF₃.

As described above, some embodiments of the present invention provide a method of fluorinating a benzoic acid or derivative compound comprising:

reacting a benzoic acid or derivative compound having the formula (I) to form a benzamide compound having the formula (II),
wherein X is at least one substitution group attached with a benzene ring and is independently selected from the group of radicals consisting of H, halo, C₁₋₂₀ alkyl, C₂₋₂₀ alkenyl, C₂₋₂₀ alkynyl, C₁₋₂₀ alkoxyl, C₆₋₂₀ aryl, C₂₋₂₀ alkylaryl, C₄₋₂₀ heterocycle, C₄₋₂₀ heteroaryl, C₄₋₂₀ alkyl heterocycle, C₇₋₂₀ alkyl heteroaryl, C₆₋₂₀ aryloxy, —OH, —CO, —COOH, —NO₂, —CN, —NH₂, —N₃, —CF₃, —OCF₃, —NR³R³′, —N(O)R³, —SH, —SR³, —SOR³, —SO₂R³, —C(O)R³, —CO₂R³, —C(O)NR₃R³′, and —OC(O)R³; wherein the alkyl, alkenyl, alkynyl, alkoxy, aryl, heterocycle, heteroaryl, or aryloxy may be substituted or unsubstituted and wherein in the alkyl portion one or more —CH₂—, CH₂CH₂—, —(CH₂)ₙ— groups are each optionally replaced by —O— or —NH—; wherein n is an integer equal to or greater than 1;
R³ and R³′ are each independently selected from the group of radicals consisting of H, C₁₋₂₀ alkyl, C₂₋₂₀ alkenyl, C₂₋₂₀ alkynyl, C₆₋₂₀ aryl; C₂₋₂₀ alkylaryl, C₄₋₂₀ heterocycle, C₄₋₂₀ heteroaryl, C₄₋₂₀ alkylheterocycle, and C₇₋₂₀ alkylheteroaryl;
wherein the alkyl, alkenyl, alkynyl, aryl, heterocycle, or heteroaryl, be substituted or unsubstituted and wherein in the alkyl portion one or more —CH₂—, CH₂CH₂—, —(CH₂)ₙ— groups are each optionally replaced by —O— or —NH—; n is an integer equal to or greater than 1;
wherein Ar is an aromatic substitution group comprising at least one fluorine atom; and

reacting the benzamide compound having the formula (II) and a mixture comprising:
a palladium (II) catalyst; and
a fluorinating reagent;
to form at least one of the ortho-fluorinated compounds having the formulae (IIa) and (IIb).
wherein X and Ar are described above.

In some embodiments, this method further comprises transforming the at least one of the ortho-fluorinated compounds having the formula (IIa) and (IIb) into an ortho-fluorinated benzoic acid or derivative compound having the formula (IIa) or (IIb).

Some embodiments of the present invention provide a method of fluorinating a benzoic acid or derivative compound comprising:

reacting a compound having the formula (II),
wherein X is at least one substitution group attached with a benzene ring and is independently selected from the group of radicals consisting of H, halo, C₁₋₂₀ alkyl, C₂₋₂₀ alkenyl, C₂₋₂₀ alkynyl, C₁₋₂₀ alkoxyl, C₆₋₂₀ aryl, C₂₋₂₀ alkylaryl, C₄₋₂₀ heterocycle, C₄₋₂₀ heteroaryl, C₄₋₂₀ alkyl heterocycle, C₇₋₂₀ alkyl heteroaryl, C₆₋₂₀ aryloxy, —OH, —CO, —COOH, —NO₂, —CN, —NH₂, —N₃, —CF₃, —OCF₃, —NR³R³′, —N(O)R³, —SH, —SR³, —SOR³, —SO₂R³, —C(O)R³, —CO₂R³, —C(O)NR₃R³′, and —OC(O)R³; wherein the alkyl, alkenyl, alkynyl, alkoxy, aryl, heterocycle, heteroaryl, or aryloxy may be substituted or unsubstituted and wherein in the alkyl portion one or more —CH₂—, CH₂CH₂—, —(CH₂)ₙ— groups are each optionally replaced by —O— or —NH—; wherein n is an integer equal to or greater than 1;
R³ and R³′ are each independently selected from the group of radicals consisting of H, C₁₋₂₀ alkyl, C₂₋₂₀ alkenyl, C₂₋₂₀ alkynyl, C₆₋₂₀ aryl; C₇₋₂₀ alkylaryl, C₄₋₂₀ heterocycle, C₄₋₂₀ heteroaryl, C₄₋₂₀ alkylheterocycle, and C₇₋₂₀ alkylheteroaryl;
wherein the alkyl, alkenyl, alkynyl, aryl, heterocycle, or heteroaryl, be substituted or unsubstituted and wherein in the alkyl portion one or more —CH₂—, CH₂CH₂—, —(CH₂)ₙ— groups are each optionally replaced by O or NH—; n is an integer equal to or greater than 1;
wherein Ar is an aromatic substitution group comprising at least one fluorine atom;

and a mixture comprising:
a palladium (II) catalyst; and
a fluorinating reagent;
to form at least one of the ortho-fluorinated compounds having the formulae (IIa) and (IIb),
wherein X and Ar are described above.

The fluorination reactions disclosed herein have wide ranges of utility allowing for the synthesis of a variety of fluorinated compounds as potential drug candidates. The methods described herein can readily provide a broad range of compounds and improve the biological properties of biologic agent.

When the following abbreviations are used throughout this disclosure, they have the following meaning:

Ac acetyl

aq aqueous

CDCl₃ deuterated chloroform

DMF dimethyl formamide

ESI electrospray (mass spectrometry)

ESI-TOF electrospray/time of flight mass spectrometry

EtOAc ethyl acetate

Hz hertz

IR infra red spectroscopy

mg milligram(s)

MHz megahertz

min minute(s)

mL milliliter

mmol millimole(s)

mol mole

MS mass spectrometry

Ms methanesulfonyl

NMP N-methylpyrrolidone

NMR nuclear magnetic resonance

Ph phenyl

ppm parts per million

rt room temperature

Tf trifluoromethanesulfonyl

TFA trifluoroacetyl

TLC thin layer chromatography

TOF time of flight (mass spectrometry)

Ts toluensulfonyl

w/w weight per unit weight

substrate a compound having formula (II) in this disclosure.

A comprehensive list of abbreviations utilized by organic chemists (i.e. persons of ordinary skill in the art) appears in the first issue of each volume of the Journal of Organic Chemistry. The list, which is typically presented in a table entitled “Standard List of Abbreviations” is incorporated herein by reference.

Embodiments of the invention may be practiced without the theoretical aspects presented. Moreover, the theoretical aspects are presented with the understanding that Applicants do not seek to be bound by the theory presented.

While various embodiments of the present invention have been described above, it should be understood that they have been presented by way of example only, and not limitation. Numerous changes to the disclosed embodiments can be made in accordance with the disclosure herein without departing from the spirit or scope of the invention. Thus, the breadth and scope of the present invention should not be limited by any of the above described embodiments.

All documents mentioned herein are incorporated herein by reference. All publications and patent documents cited in this application are incorporated by reference for all purposes to the same extent as if each individual publication or patent document were so individually denoted. By their citation of various references in this document, applicants do not admit any particular reference is “prior art” to their invention.

Embodiments of inventive compositions and methods are illustrated in the following examples.

### EXAMPLES

### Materials and Methods

Solvents were obtained from Sigma-Aldrich, Alfa-Aesar, Acros and EMD Chemicals and used directly without further purification. Palladium catalysts were obtained from Sigma-Aldrich and Pressure Chemical Company. Carboxylic acids or carboxylic acid chlorides were obtained from Sigma-Aldrich, Alfa-Aesar, Acros, Oakwood and Strem Chemicals, and used to prepare corresponding amides. Octafluorotoluene was purchased from Oakwood and fluorinating reagents (F1-F5) were purchased from TCI America and Sigma-Aldrich.

Analytical thin layer chromatography was performed on 0.25 mm silica gel 60-F254. Preparative thin layer chromatography was performed on 0.5 mm silica gel purchased from Analtech. Visualization was carried out with UV light and Vogel's permanganate. NMR spectra were recorded on a Bruker AMX-400 instrument (400 MHz for ¹H; 100 MHz for ¹³C; 375 MHz for 19F), Varian Inova-400 instrument (400 MHz for ¹H; 100 MHz for ¹³C), Bruker DRX-500 instrument (500 MHz for 1H; 125 MHz for ¹³C), or Bruker DRX-600 instrument (600 MHz for ¹H; 150 MHz for ¹³C) equipped with a 5 mm DCH cryoprobe. Calibration was done using residual undeuterated solvent or SiMe₄ as an internal reference for ¹H and ¹³C. CCl3F was used as an external reference for 19F. The following abbreviations (or combinations thereof) were used to explain multiplicities: s=singlet, d=doublet, t=triplet, m=multiplet, br=broad. High-resolution mass spectra (HRMS) were recorded on an Agilent Mass spectrometer using ESI-TOF (electrospray ionization-time of flight). IR spectra were recorded on a Perkin Elmer Spectrum BX FTIR spectrometer.

Substrate Structures:

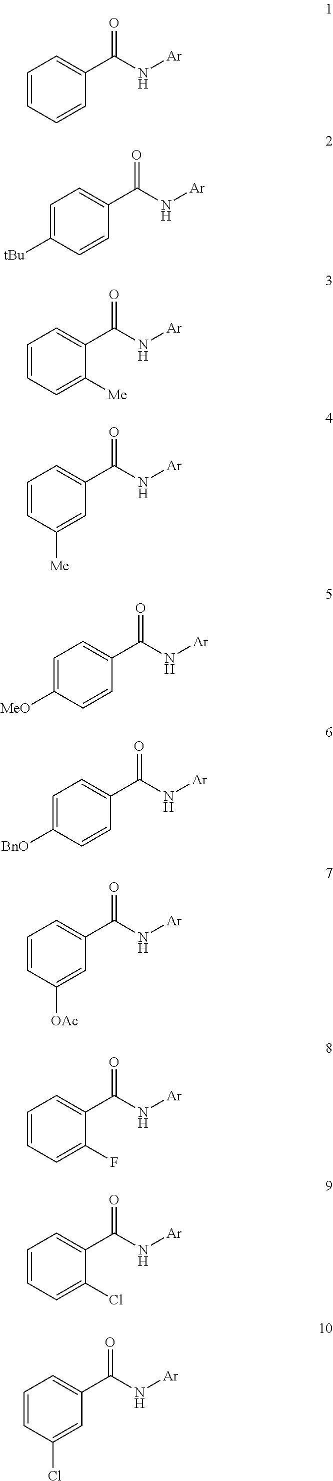

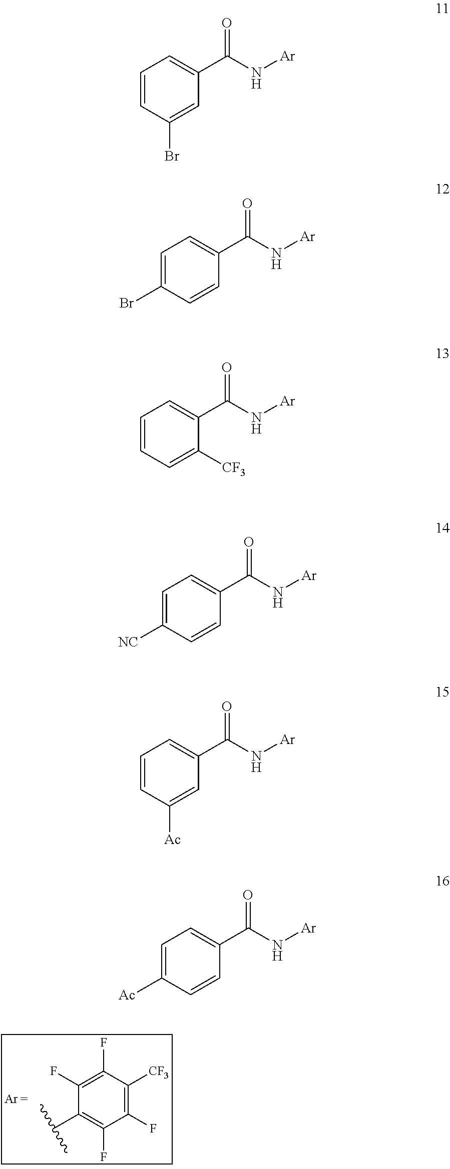

### Example 1

### Substrate Preparation

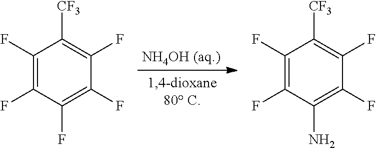 [SMILES: Fc1c(F)c(F)c(C(F)(F)F)c(F)c1F ; Nc1c(F)c(F)c(C(F)(F)F)c(F)c1F]

### Preparation of 2,3,5,6-Tetrafluoro-4-(triluoromethyl)aniline

^{[1]} To a solution of octafluorotoluene (30 mL) in 1,4-dioxane (79 mL) was added aq. NH₄OH (53 mL, 28% w/w), and the reaction flask was sealed and heated to 80° C. The reaction mixture was stirred until the substrate was completely consumed, as verified by GCMS and TLC. The mixture was then cooled, and 1,4-dioxane was removed in vacuo. The resultant mixture was extracted with ethyl acetate (3×100 mL), and then concentrated in vacuo. The crude mixture was purified by column chromatography (hexanes→hexanes:ethyl acetate=4:1) to afford 2,3,5,6-tetrafluoro-4-(trifluoromethyl)aniline as a colorless liquid (72 g, 72%).

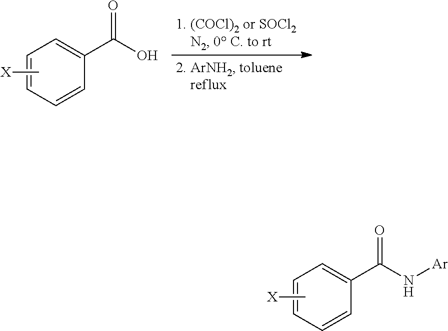

General Procedure for the Preparation of Benzamides:

An acid chloride (20 mmol), prepared from the corresponding carboxylic acid and oxalyl chloride or thionyl chloride, was added to a vigorously stirred solution of 2,3,5,6-tetrafluoro-4-(trifluoromethyl)aniline (21 mmol) in toluene (3 mL). The reaction mixture was stirred for 24 h under reflux, and then stirred at room temperature for 4 h. The product mixture was concentrated in vacuo and was recrystallized from ethyl acetate/hexane (100° C. to −30° C.) to give the amide. The prepared substrates were used without further purification provided that they appeared pure by TLC, GCMS and NMR. The directing group peaks in the ¹³C NMR spectra were not reported due to the complex splitting of the corresponding peaks from C—F coupling. However, the presence of F atoms was verified by the ¹⁹F NMR spectra.

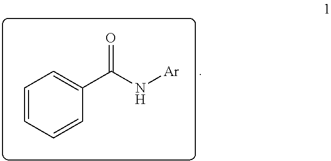

### N-(2,3,5,6-Tetrafluoro-4-(trifluoromethyl)phenyl)benzamide (1)

¹H NMR (400 MHz, (CD₃)₂SO) δ 10.92 (s, 1H), 8.02 (d, J=8.3 Hz, 2H), 7.67 (t, J=7.4 Hz, 1H), 7.58 (t, J=7.6 Hz, 2H); ¹³C NMR (100 MHz, (CD₃)₂SO)S 165.15, 132.80, 132.13, 128.71, 128.22; ¹⁹F NMR (375 MHz, (CD₃)₂SO) δ −54.75 (t, J=20.7 Hz, 3F), −142.06 to −142.34 (m, 4F); HRMS (ESI-TOF) Calcd for C₁₄H₇F₇NO (MH⁺): 338.0410. found: 338.0423.

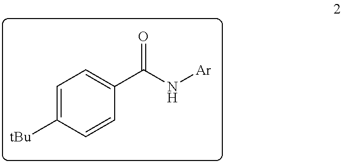

### 4-(tert-Butyl)-N-(2,3,5,6-tetrafluoro-4-(trifluoromethyl)phenyl)benzamide (2)

¹H NMR (400 MHz, CDCl₃) δ 7.87 (d, J=8.3 Hz, 1H), 7.69 (s, 1H), 7.54 (d, J=8.3 Hz, 1H), 1.36 (s, 9H); ¹³C NMR (100 MHz, CDCl₃) δ 164.89, 157.29, 129.24, 127.90, 126.17, 35.33, 31.22; HRMS (ESI-TOF) Calcd for C₁₈H₁₅F₇NO (MH⁺): 394.1036. found: 394.1051.

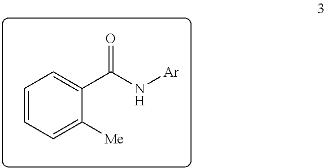

### 2-Methyl-N-(2,3,5,6-tetrafluoro-4-(trifluoromethyl)phenyl)benzamide (3)

¹H NMR (400 MHz, (CD₃)₂SO) δ 10.94 (s, 1H), 7.54 (d, J=7.6 Hz, 1H), 7.48 to 7.44 (m, 1H), 7.37 to 7.33 (m, 2H), 2.42 (s, 3H); ¹³C NMR (100 MHz, (CD₃)₂SO) δ 167.41, 136.03, 134.48, 130.88, 130.71, 127.74, 125.82, 19.41; HRMS (ESI-TOF) Calcd for C₁₅H₉F₇NO (MH⁺): 352.0567. found: 352.0581.

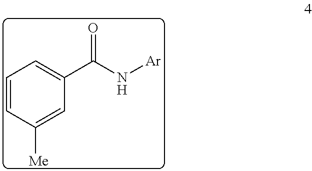

### 3-Methyl-N-(2,3,5,6-tetrafluoro-4-(trifluoromethyl)phenyl)benzamide (4)

¹H NMR (400 MHz, (CD₃)₂SO) δ 10.87 (s, 1H), 7.84 (s, 1H), 7.81 (d, J=7.0 Hz, 1H), 7.50 to 7.44 (m, 2H), 2.41 (s, 3H); ¹³C NMR (100 MHz, (CD₃)₂SO) δ 165.22, 138.15, 133.37, 132.10, 128.67, 128.61, 125.36, 20.89; HRMS (ESI-TOF) Calcd for C₁₅H₉F₇NO (MH⁺): 352.0567. found: 352.0584.

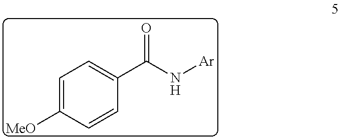

### 4-Methoxy-N-(2,3,5,6-tetrafluoro-4-(trifluoromethyl)phenyl)benzamide (5)

¹H NMR (400 MHz, (CD₃)₂SO) δ 10.67 (s, 1H), 8.01 (d, J=8.9 Hz, 2H), 7.10 (d, J=8.9 Hz, 2H), 3.86 (s, 3H); ¹³C NMR (100 MHz, (CD₃)₂SO) δ 164.43, 162.79, 130.28, 124.19, 113.91, 55.52; HRMS (ESI-TOF) Calcd for C₁₅H₉F₇NO₂(MH⁺): 368.0516. found: 368.0528.

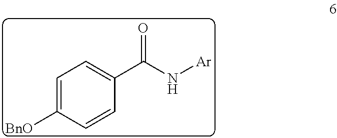

### 4-(Benzyloxy)-N-(2,3,5,6-tetrafluoro-4-(trifluoromethyl)phenyl)benzamide (6)

¹H NMR (600 MHz, CD₃OD) δ 7.98 (d, J=8.8 Hz, 2H), 7.46 (d, J=7.4 Hz, 2H), 7.39 (t, J=7.5 Hz, 2H), 7.33 (t, J=7.3 Hz, 1H), 7.15 (d, J=8.8 Hz, 2H), 5.20 (s, 2H); ¹³C NMR (150 MHz, CD₃OD) δ 167.73, 164.08, 138.02, 131.31, 129.61, 129.14, 128.70, 125.88, 115.99, 71.25.

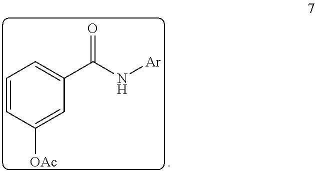

### 3-((2,3,5,6-Tetrafluoro-4-(trifluoromethyl)phenyl)carbamoyl)phenyl acetate (7)

¹H NMR (600 MHz, (CD₃)₂SO) δ 7.93 (d, J=7.4 Hz, 1H), 7.77 (s, 1H), 7.64 (t, J=7.8 Hz, 1H), 7.46 (d, J=7.6 Hz, 1H), 2.31 (s, 3H); ¹³C NMR (150 MHz, (CD₃)₂SO) δ 169.23, 164.20, 150.58, 133.47, 130.04, 126.48, 125.63, 121.74, 20.82.

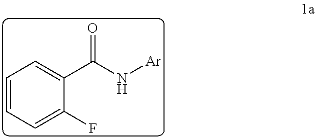

### 2-Fluoro-N-(2,3,5,6-tetrafluoro-4-(trifluoromethyl)phenyl)benzamide (1a)

¹H NMR (600 MHz, (CD₃)₂SO) δ 7.74 (dt, J=7.5, 1.7 Hz, 1H), 7.68 to 7.65 (m, 1H), 7.42 to 7.36 (m, 2H); ¹³C NMR (100 MHz, (CD₃)₂SO) δ 162.59, 159.36 (d, J=251.2 Hz), 133.84 (d, J=8.6 Hz), 130.30 (d, J=2.1 Hz), 124.73 (d, J=3.5 Hz), 122.21 (d, J=13.9 Hz), 116.43 (d, J=21.6 Hz); ¹⁹F NMR (375 MHz, (CD₃)₂SO) δ −54.86 (t, J=20.8 Hz, 3F), −112.93 (s, 1F), −141.93 to −142.28 (m, 4F); IR (neat) ν 3255, 1685, 1477, 1349, 1238, 1144, 1136, 1003, 995, 908, 754, 720 cm⁻¹; HRMS (ESI-TOF) Calcd for C₁₄H₆F₈NO (MH⁺): 356.0316. found: 356.0322.

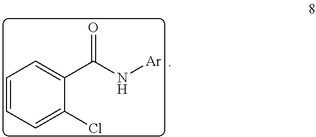

### 2-Chloro-N-(2,3,5,6-tetrafluoro-4-(trifluoromethyl)phenyl)benzamide (8)

¹H NMR (400 MHz, CD₃OD) δ 7.61 (ddd, J=7.5, 1.6, 0.6 Hz, 1H), 7.55 to 7.49 (m, 2H), 7.44 (ddd, J=7.5, 6.7, 2.0 Hz, 1H); ¹³C NMR (150 MHz, (CD₃)₂SO) δ 164.79, 134.75, 132.05, 130.18, 130.00, 129.29, 127.42.

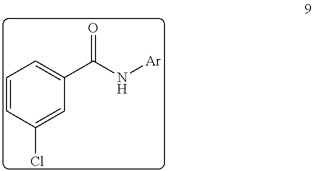

### 3-Chloro-N-(2,3,5,6-tetrafluoro-4-(trifluoromethyl)phenyl)benzamide (9)

¹H NMR (400 MHz, (CD₃)₂SO) δ 11.06 (s, 1H), 8.06 (s, 1H), 7.97 (d, J=7.8 Hz, 1H), 7.74 (dd, J=8.0, 1.0 Hz, 1H), 7.61 (t, J=7.9 Hz, 1H); ¹³C NMR (100 MHz, (CD₃)₂SO) δ 163.79, 134.03, 133.53, 132.63, 130.75, 127.96, 127.00; HRMS (ESI-TOF) Calcd for C₁₄H₆ClF₇NO (MH⁺): 372.0021. found: 372.0029.

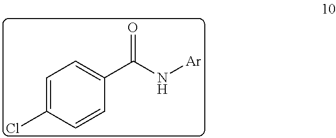

### 4-Chloro-N-(2,3,5,6-tetrafluoro-4-(trifluoromethyl)phenyl)benzamide (10)

¹H NMR (400 MHz, (CD₃)₂SO) δ 11.02 (br s, 1H), 8.03 (d, J=8.5 Hz, 2H), 7.66 (d, J=8.5 Hz, 2H); ¹³C NMR (100 MHz, (CD₃)₂SO)S 164.16, 137.71, 130.84, 130.13, 128.84; HRMS (ESI-TOF) Calcd for C₁₄H₆ClF₇NO (MH⁺): 372.0021. found: 372.0039.

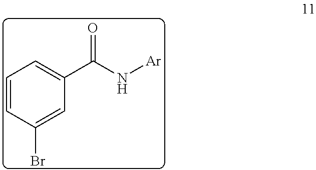

### 3-Bromo-N-(2,3,5,6-tetrafluoro-4-(trifluoromethyl)phenyl)benzamide (11)

¹H NMR (400 MHz, (CD₃)₂SO) δ 11.06 (s, 1H), 8.19 (s, 1H), 8.00 (d, J=7.9 Hz, 1H), 7.87 (d, J=8.0 Hz, 1H), 7.54 (t, J=7.9 Hz, 1H); ¹³C NMR (100 MHz, (CD₃)₂SO)S 163.70, 135.53, 134.21, 130.98, 130.81, 127.38, 121.91; HRMS (ESI-TOF) Calcd for C₁₄H₆BrF₇NO (MH⁺): 415.9515. found: 415.9535.

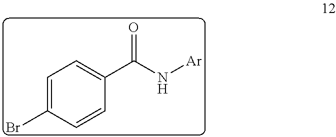

### 4-Bromo-N-(2,3,5,6-tetrafluoro-4-(trifluoromethyl)phenyl)benzamide (12)

¹H NMR (400 MHz, (CD₃)₂SO) δ 11.02 (s, 1H), 7.95 (d, J=8.6 Hz, 2H), 7.80 (d, J=8.6 Hz, 2H); ¹³C NMR (100 MHz, (CD₃)₂SO) δ 164.33, 131.83, 131.21, 130.28, 126.78; HRMS (ESI-TOF) Calcd for C₁₄H₆BrF₇NO (MH⁺): 415.9515. found: 415.9533.

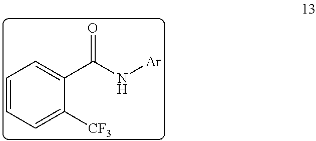

### N-(2,3,5,6-Tetrafluoro-4-(trifluoromethyl)phenyl)-2-(trifluoromethyl)benzamide (13)

¹H NMR (600 MHz, (CD₃)₂SO) δ 7.90 (d, J=7.8 Hz, 1H), 7.85 (t, J=7.4 Hz, 1H), 7.78 (t, J=7.6 Hz, 1H), 7.74 (d, J=7.5 Hz, 1H); ¹³C NMR (150 MHz, (CD₃)₂SO) δ 165.56, 133.97, 132.79, 130.99, 128.83, 126.64 (q, J=4.6 Hz), 126.29 (q, J=31.7 Hz), 123.54 (q, J=273.6 Hz); ¹⁹F NMR (375 MHz, (CD₃)₂SO) δ −54.88 (t, J=21.2 Hz, 3F), −57.53 (s, 3F), −141.88 to −142.16 (m, 2F), −142.44 to −142.48 (m, 2F).

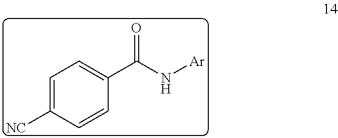

### 4-Cyano-N-(2,3,5,6-tetrafluoro-4-(trifluoromethyl)phenyl)benzamide (14)

¹H NMR (400 MHz, (CD₃)₂SO) δ 11.22 (s, 1H), 8.15 (d, J=8.3 Hz, 2H), 8.07 (d, J=8.4 Hz, 2H); ¹³C NMR (100 MHz, (CD₃)₂SO) δ 163.97, 136.02, 132.80, 129.03, 118.13, 115.04; HRMS (ESI-TOF) Calcd for C₁₅H₆F₇N₂O (MH⁺): 363.0363. found: 363.0367.

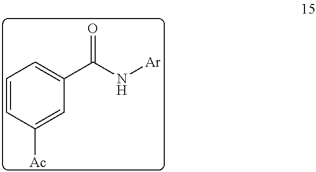

### 3-Acetyl-N-(2,3,5,6-tetrafluoro-4-(trifluoromethyl)phenyl)benzamide (15)

¹H NMR (400 MHz, CD₃OD) δ 8.60 (dt, J=1.8, 0.5 Hz, 1H), 8.26 to 8.20 (m, 2H), 7.69 (dt, J=7.8, 0.5 Hz, 1H), 2.67 (s, 3H); ¹³C NMR (100 MHz, CD₃OD) δ 199.17, 167.23, 138.83, 134.30, 133.62, 133.49, 130.41, 128.95, 26.77.

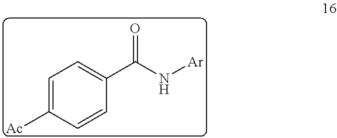

### 4-Acetyl-N-(2,3,5,6-tetrafluoro-4-(trifluoromethyl)phenyl)benzamide (16)

¹H NMR (400 MHz, (CD₃)₂SO) δ 11.13 (s; 1H), 8.13 (s, 4H), 2.65 (s, 3H); ¹³C NMR (100 MHz, (CD₃)₂SO) δ 197.71, 164.49, 139.73, 135.76, 128.58, 128.45, 27.07; HRMS (ESI-TOF) Calcd for C₁₆H₉F₇NO₂ (MH⁺): 380.0516. found: 380.0521.

### Example 2

### Synthesis of Pd(OTf)₂(MeCN)₄^{[2]}

To a dark orange solution of palladium (II) acetate (2.04 g, 9.09 mmol) in acetonitrile (172 mL), neat trifluoromethanesulfonic acid (2.35 mL, 26.52 mmol) was added dropwise while stirring. The reaction mixture subsequently changed to a pale yellow color. Diethyl ether (240 mL) was then added, and a fine pale yellow powder precipitated. The suspension was set aside for 2 h and the supernatant liquid was decanted. The residual powder was washed with diethyl ether and dried under a stream of nitrogen to afford Pd(OTf)₂(MeCN)₄ as a pale yellow powder (3.75 g, 73%).

### Example 3

### Palladium(II)-Catalyzed Fluorination of Benzoic Acids Using a Practical Auxiliary

Optimization of Reaction Conditions^{[a,b]}

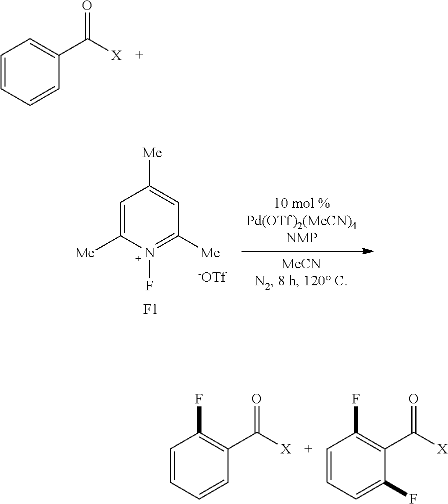

TABLE 1

| Entry | X | mono (%) | di (%) | SM (%) |
|---|---|---|---|---|
| 1 | 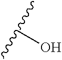 [SMILES: *O] | 0 | 0 | 100 |
| 2 | 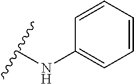 [SMILES: *Nc1ccccc1] | 0 | 0 | 85 |
| 3 | 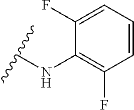 [SMILES: *Nc1c(F)cccc1F] | 50 | 0 | 50 |
| 4 | 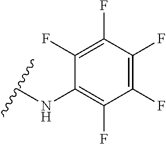 [SMILES: *Nc1c(F)c(F)c(F)c(F)c1F] | 78 | 1 | 21 |
| 5 | 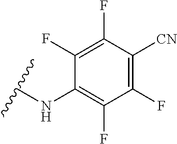 [SMILES: *Nc1c(F)c(F)c(C#N)c(F)c1F] | 84 | 2 | 9 |
| 6 | 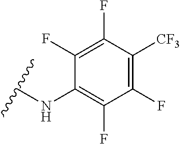 [SMILES: *Nc1c(F)c(F)c(C(F)(F)F)c(F)c1F] | 84 | 4 | 7 |

[a] Conditions: 0.1 mmol of substrate, 10 mol % Pd(OTf)₂(MeCN)₄, 20 mol % N-methyl-2-pyrrolidone (NMP), 1.5 equiv of N-fluoro-2,4,6-trimethylpyridinium triflate (F1), 2 mL MeCN, 120° C., N₂, 8 h. [b] Yield was determined by ¹H NMR analysis of the crude reaction mixture in CDCl₃ using CH₂Br₂ as the internal standard.

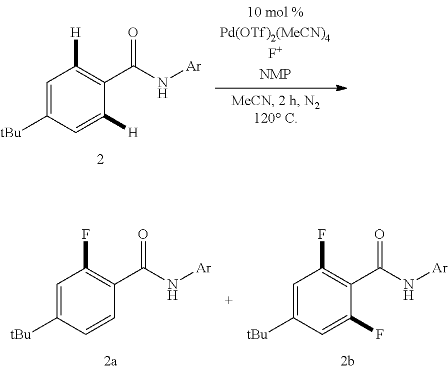

TABLE 2

| Entry | F⁺ | | 2a (%) | 2b (%) | 2 (%) |
|---|---|---|---|---|---|
| 1 | 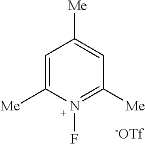 | F1 | 82 | 5 | 11 |
| 2 | 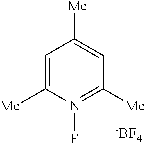 | F2 | 63 | 2 | 27 |
| 3 | 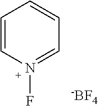 | F3 | 34 | 0 | 66 |
| 4 | 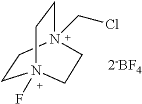 | F4 | 70 | 0 | 30 |
| 5 | 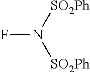 | F5 | 60 | 0 | 40 |

[a] Conditions: 0.1 mmol of substrate, 10 mol % Pd(OTf)₂(MeCN)₄, 20 mol % N-methyl-2-pyrrolidone (NMP), 1.5 equiv of F⁺ reagent, 2 mL MeCN, 120° C., N₂, 2 h. [b] Yield was determined by ¹H NMR analysis of the crude reaction mixture in CDCl₃ using CH₂Br₂ as the internal standard.

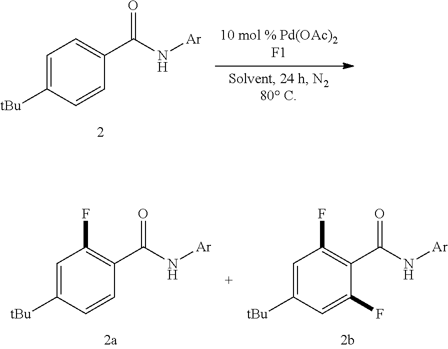

TABLE 3

| Entry | Solvent | 2a (%) | 2b (%) | 2 (%) |
|---|---|---|---|---|
| 1 | PhCF₃ | 0 | 50 | 0 |
| 2 | iPr₂O | 0 | 0 | 89 |
| 3 | tAmylOH | 0 | 0 | 98 |
| 4 | tBuOH | 0 | 0 | 98 |
| 5 | C₆F₆ | 1 | 0 | 84 |
| 6 | 1,4-dioxane | 0 | 13 | 65 |
| 7 | DMF | 0 | 0 | 100 |
| 8 | DMA | 0 | 4 | 88 |
| 9 | THF | 0 | 0 | 95 |
| 10 | EtOAc | 0 | 29 | 0 |
| 11 | tBuOAc | 0 | 0 | 97 |
| 12 | n-hexane | 0 | 0 | 95 |
| 13 | NMP | 1 | 1 | 87 |
| 14 | cyclohexane | 0 | 0 | 73 |
| 15 | pinacolone | 0 | 0 | 100 |
| 16 | MeCN | 54 | 2 | 11 |

[a] Conditions: 0.1 mmol of substrate, 10 mol % Pd(OAc)₂, 3.0 equiv of N-fluoro-2,4,6-trimethylpyridinium triflate (F1), 1 mL solvent, 80° C., N₂, 24 h. [b] Yield was determined by ¹H NMR analysis of the crude reaction mixture in CDCl₃ using CH₂Br₂ as the internal standard.

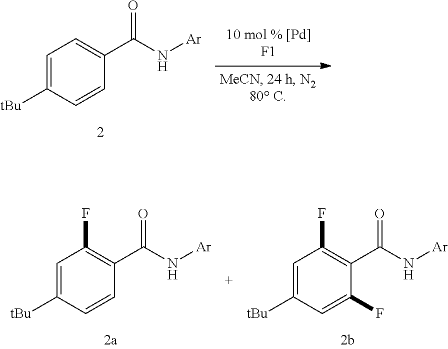

TABLE 4

| Entry | [Pd] | 2a (%) | 2b (%) | 2 (%) |
|---|---|---|---|---|
| 1 | Pd(OPiv)₂ | 57 | 11 | 0 |
| 2 | Pd₂(dba)₃ | 61 | 5 | 5 |
| 3 | PdCl₂ | 0 | 0 | 100 |
| 4 | PdCl₂(MeCN)₂ | 0 | 0 | 100 |
| 5 | Pd(TFA)₂ | 63 | 4 | 7 |
| 6 | Pd(OTf)₂(MeCN)₄ | 65 | 3 | 7 |
| 7 | Pd(OTf)₂(H₂O)₂ | 60 | 3 | 8 |
| 8 | Pd/C (10% wt) | 0 | 0 | 100 |
| 9 | Pd(OAc)₂ | 54 | 2 | 11 |

[a] Conditions: 0.1 mmol of substrate, 10 mol % [Pd] catalyst, 3.0 equiv. of N-fluoro-2,4,6-trimethylpyridinium triflate (F1), 1 mL MeCN, 80° C., N₂, 24 h. [b] Yield was determined by ¹H NMR analysis of the crude reaction mixture in CDCl₃ using CH₂Br₂ as the internal standard.

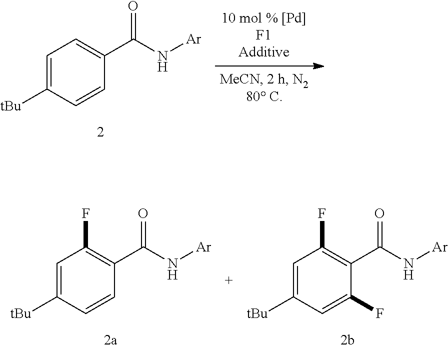

TABLE 5

| Entry | [Pd] | Additive | 2a (%) | 2b (%) | 2 (%) |
|---|---|---|---|---|---|
| 1 | Pd(OPiv)₂ | — | 29 | 0 | 64 |
| 2 | Pd(OPiv)₂ | NMP | 38 | 2 | 49 |
| 3 | Pd(OPiv)₂ | EtOAc | 41 | 1 | 50 |
| 4 | Pd(OPiv)₂ | DMF | 23 | 1 | 51 |
| 5 | Pd(OPiv)₂ | DMA | 38 | 3 | 51 |
| 6 | Pd₂(dba)₃ | — | 32 | 1 | 55 |
| 7 | Pd₂(dba)₃ | NMP | 46 | 0 | 38 |
| 8 | Pd₂(dba)₃ | EtOAc | 24 | 0 | 61 |
| 9 | Pd₂(dba)₃ | DMF | 36 | 1 | 48 |
| 10 | Pd₂(dba)₃ | DMA | 26 | 1 | 59 |
| 11 | Pd(TFA)₂ | — | 44 | 1 | 47 |
| 12 | Pd(TFA)₂ | NMP | 36 | 1 | 59 |
| 13 | Pd(TFA)₂ | EtOAc | 42 | 1 | 53 |
| 14 | Pd(TFA)₂ | DMF | 31 | 1 | 59 |
| 15 | Pd(TFA)₂ | DMA | 34 | 1 | 59 |
| 16 | Pd(OTf)₂(MeCN)₄ | — | 32 | 0 | 68 |
| 17 | Pd(OTf)₂(MeCN)₄ | NMP | 51 | 1 | 40 |
| 18 | Pd(OTf)₂(MeCN)₄ | EtOAc | 34 | 0 | 57 |
| 19 | Pd(OTf)₂(MeCN)₄ | DMF | 32 | 0 | 57 |
| 20 | Pd(OTf)₂(MeCN)₄ | DMA | 33 | 0 | 53 |
| 21 | Pd(OTf)₂(H₂O)₂ | — | 36 | 0 | 64 |
| 22 | Pd(OTf)₂(H₂O)₂ | NMP | 48 | 1 | 38 |
| 23 | Pd(OTf)₂(H₂O)₂ | EtOAc | 36 | 0 | 64 |
| 24 | Pd(OTf)₂(H₂O)₂ | DMF | 35 | 0 | 55 |
| 25 | Pd(OTf)₂(H₂O)₂ | DMA | 28 | 0 | 57 |

[a] Conditions: 0.1 mmol of substrate, 10 mol % [Pd] catalyst, 20 mol % additive, 3.0 equiv. of N-fluoro-2,4,6-trimethylpyridinium triflate (F1), 1 mL MeCN, 80° C., N₂, 2 h. [b] Yield was determined by ¹H NMR analysis of the crude reaction mixture in CDCl₃ using CH₂Br₂ as the internal standard.

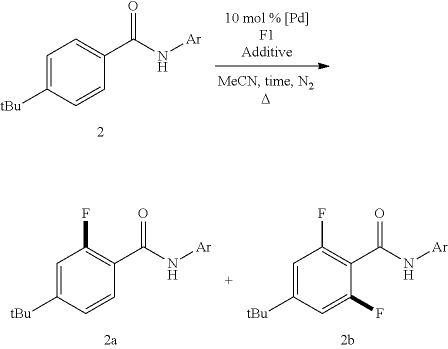

TABLE 6

| Entry | [Pd] | Additive | Time (h) | Temp (° C.) | 2a (%) | 2b (%) | 2 (%) |
|---|---|---|---|---|---|---|---|
| 1 | Pd(OPiv)₂ | NMP (20 mol %) | 2 | 100 | 49 | 2 | 31 |
| 2 | Pd(OPiv)₂ | EtOAc (20 mol %) | 2 | 100 | 46 | 1 | 47 |
| 3 | Pd(OPiv)₂ | DMA (20 mol %) | 2 | 100 | 50 | 4 | 31 |
| 4 | Pd(TFA)₂ | — | 2 | 100 | 51 | 1 | 47 |
| 5 | Pd(TFA)₂ | EtOAc (20 mol %) | 2 | 100 | 71 | 6 | 21 |
| 6 | Pd(TFA)₂ | — | 2 | 120 | 70 | 11 | 8 |
| 7 | Pd(TFA)₂ | EtOAc (20 mol %) | 2 | 120 | 77 | 12 | 10 |
| 8 | Pd(OTf)₂(MeCN)₄ | NMP (20 mol %) | 2 | 100 | 76 | 6 | 12 |
| 9 | Pd(OTf)₂(MeCN)₄ | NMP (20 mol %) | 3 | 100 | 74 | 6 | 9 |
| 10 | Pd(OTf)₂(MeCN)₄ | NMP (20 mol %) | 4 | 100 | 74 | 15 | 0 |
| 11 | Pd(OTf)₂(MeCN)₄ | NMP (10 mol %) | 2 | 100 | 65 | 2 | 26 |
| 12 | Pd(OTf)₂(MeCN)₄ | NMP (20 mol %) | 2 | 120 | 71 | 7 | 12 |
| 13 | Pd(OTf)₂(H₂O)₂ | NMP (20 mol %) | 2 | 100 | 75 | 4 | 18 |
| 14 | Pd(OTf)₂(H₂O)₂ | NMP (20 mol %) | 3 | 100 | 70 | 4 | 11 |
| 15 | Pd(OTf)₂(H₂O)₂ | NMP (20 mol %) | 4 | 100 | 75 | 10 | 0 |
| 16 | Pd(OTf)₂(H₂O)₂ | NMP (10 mol %) | 2 | 100 | 57 | 2 | 30 |
| 17 | Pd(OTf)₂(H₂O)₂ | NMP (20 mol %) | 2 | 120 | 71 | 6 | 11 |

[a] Conditions: 0.1 mmol of substrate, 10 mol % [Pd] catalyst, additive, 3.0 equiv. of N-fluoro-2,4,6-trimethylpyridinium triflate (F1), 1 mL MeCN, temp, N₂, time. [b] Yield was determined by ¹H NMR analysis of the crude reaction mixture in CDCl₃ using CH₂Br₂ as the internal standard.

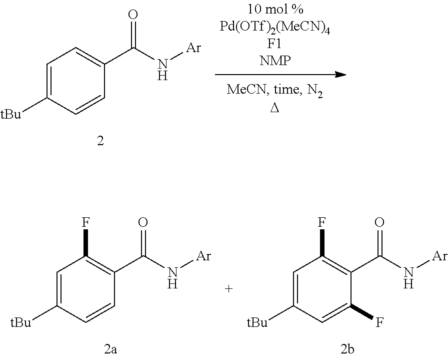

TABLE 7

| | Vol | F1 | Temp | Time | 2a | 2b | 2 |
|---|---|---|---|---|---|---|---|
| Entry | (mL) | (eq.) | (° C.) | (h) | (%) | (%) | (%) |
| 1 | 0.5 | 1.5 | 100 | 2 | 70 | 2 | 12 |
| 2 | 0.5 | 1.5 | 120 | 2 | 80 | 9 | 0 |
| 3 | 0.5 | 3.0 | 100 | 2 | 72 | 9 | 9 |
| 4 | 0.5 | 3.0 | 120 | 2 | 51 | 29 | 0 |
| 5 | 0.75 | 1.0 | 100 | 2 | 62 | 0 | 29 |
| 6 | 0.75 | 1.2 | 100 | 2 | 61 | 0 | 34 |
| 7 | 0.75 | 1.2 | 100 | 3 | 68 | 1 | 25 |
| 8 | 0.75 | 1.2 | 100 | 4 | 76 | 2 | 12 |
| 9 | 0.75 | 1.5 | 100 | 2 | 86 | 2 | 0 |
| 10 | 0.75 | 1.5 | 120 | 2 | 85 | 7 | 0 |
| 11 | 0.75 | 3.0 | 100 | 2 | 70 | 11 | 5 |
| 12 | 0.75 | 3.0 | 120 | 2 | 61 | 28 | 0 |
| 13 | 1.0 | 1.0 | 100 | 2 | 68 | 0 | 32 |
| 14 | 1.0 | 1.2 | 100 | 2 | 64 | 0 | 36 |
| 15 | 1.0 | 1.2 | 100 | 3 | 74 | 2 | 20 |
| 16 | 1.0 | 1.2 | 100 | 4 | 50 | 1 | 24 |
| 17 | 2.0 | 1.5 | 100 | 2 | 73 | 1 | 25 |
| 18 | 2.0 | 1.5 | 100 | 3 | 79 | 3 | 18 |
| 19 | 2.0 | 1.5 | 100 | 4 | 70 | 2 | 23 |
| 20 | 2.0 | 1.5 | 120 | 2 | 82 | 5 | 11 |
| 21 | 2.0 | 1.5 | 120 | 3 | 79 | 7 | 11 |
| 22 | 2.0 | 1.5 | 120 | 4 | 77 | 8 | 8 |
| 23 | 2.0 | 3.0 | 100 | 2 | 73 | 4 | 21 |
| 24 | 2.0 | 3.0 | 100 | 3 | 81 | 8 | 11 |
| 25 | 2.0 | 3.0 | 100 | 4 | 87 | 13 | 0 |
| 26 | 2.0 | 3.0 | 120 | 2 | 79 | 13 | 8 |
| 27 | 3.0 | 3.0 | 100 | 2 | 75 | 3 | 22 |
| 28 | 3.0 | 3.0 | 100 | 3 | 87 | 13 | 0 |
| 29 | 3.0 | 3.0 | 100 | 4 | 80 | 10 | 10 |
| 30 | 3.0 | 3.0 | 120 | 2 | 81 | 12 | 7 |

[a] Conditions: 0.1 mmol of substrate, 10 mol % Pd(OTf)₂(MeCN)₄, 20 mol % N-methyl-2-pyrrolidone (NMP), N-fluoro-2,4,6-trimethylpyridinium triflate (F1), MeCN, temp, N₂, time. [b] Yield was determined by ¹H NMR analysis of the crude reaction mixture in CDCl₃ using CH₂Br₂ as the internal standard.

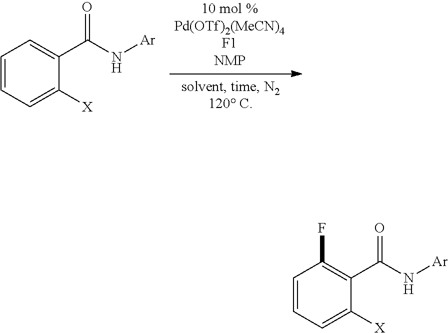

TABLE 8

| | | | Vol | F1 | NMP | Time | product | SM |
|---|---|---|---|---|---|---|---|---|
| Entry | X | Solvent | (mL) | (eq.) | (eq.) | (h) | (%) | (%) |
| 1 | F | MeCN | 2.0 | 1.5 | 0.2 | 24 | 48 | 52 |
| 2 | F | PhCF₃ | 1.0 | 1.5 | 0.5 | 2 | 72 | 1 |
| 3 | CF₃ | MeCN | 2.0 | 3.0 | 0.2 | 24 | 50 | 50 |
| 4 | CF₃ | PhCF₃ | 1.0 | 1.5 | 0.5 | 2 | 75 | 0 |

[a] Conditions: 0.1 mmol of substrate, 10 mol % Pd(OTf)₂(MeCN)₄, N-methyl-2-pyrrolidone (NMP), N-fluoro-2,4,6-trimethylpyridinium triflate (F1), solvent, 120° C., N₂, time. [a] Yield was determined by ¹H NMR analysis of the crude reaction mixture in CDCl₃ using CH₂Br₂ as the internal standard.

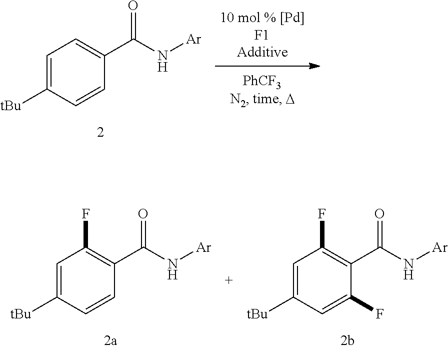

TABLE 9

| Entry | [Pd] | Additive | Temp (° C.) | Vol (ml) | Time (h) | mono (%) | di (%) | SM (%) |
|---|---|---|---|---|---|---|---|---|
| 1 | Pd(OAc)₂ | NMP | 100 | 0.5 | 2 | 0 | 37 | 0 |
| 2 | Pd(OAc)₂ | — | 100 | 0.5 | 2 | 8 | 21 | 8 |
| 3 | Pd(OAc)₂ | NMP | 100 | 1.0 | 2 | 0 | 50 | 0 |
| 4 | Pd(OAc)₂ | NMP | 100 | 1.5 | 2 | 5 | 46 | 0 |
| 5 | Pd(OAc)₂ | DMF | 100 | 0.5 | 2 | 10 | 21 | 3 |
| 6 | Pd(OAc)₂ | K₂HPO₄ | 100 | 0.5 | 2 | 7 | 7 | 39 |
| 7 | Pd(OAc)₂ | NMP | 100 | 1.0 | 1 | 6 | 47 | 1 |
| 8 | Pd(OAc)₂ | NMP | 100 | 1.0 | 0.5 | 4 | 46 | 2 |
| 9 | Pd(OTf)₂(MeCN)₄ | NMP | 100 | 1.0 | 2 | 20 | 60 | 0 |
| 10 | Pd(OTf)₂(MeCN)₄ | NMP | 120 | 1.0 | 2 | 0 | 90 | 0 |

[a] Conditions: 0.1 mmol of substrate, 10 mol % [Pd] catalyst, 50 mol % additive, 3.0 equiv. of N-fluoro-2,4,6-trimethylpyridinium triflate (F1), PhCF₃, temp, N₂, time. [b] Yield was determined by ¹H NMR analysis of the crude reaction mixture in CDCl₃ using CH₂Br₂ as the internal standard.

### Example 4

### General Reaction Scheme

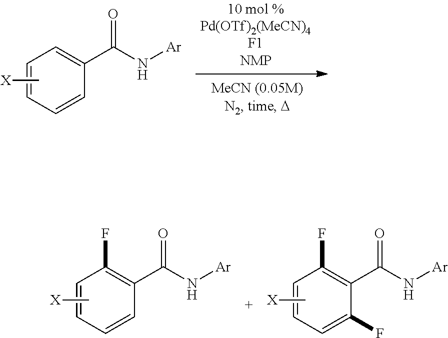

General Procedure A:

In a 50 mL Schlenk tube, benzamide (1-12 or 14-16) (0.1 mmol), Pd(OTf)₂(MeCN)₄ (5.7 mg, 0.01 mmol), N-fluoro-2,4,6-trimethylpyridinium triflate (43.4 mg, 0.15 mmol or 86.8 mg, 0.30 mmol), NMP (2.0 mg, 0.02 mmol) were dissolved in dry acetonitrile (2.0 mL) under N₂. The tube was sealed with a Teflon lined cap and the reaction mixture was heated. Although the reaction was selective for mono-fluorination, optimization was carried out to maximize conversion and material balance. After cooling to room temperature, the mixture was concentrated in vacuo and the residue was purified by column chromatography on silica gel with a gradient eluent of hexane and ethyl acetate to afford the product (1a-12a or 14a-16a, respectively). Following purification, a mixture of starting material and fluorinated products was obtained. In each case, the ratio (starting material/product) was determined by analysis of the ¹H NMR spectrum. Isolated yields were reported as averages of 5 runs.

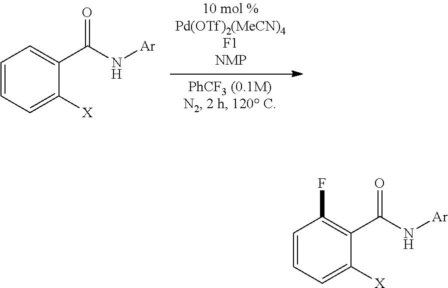

General Procedure B:

In a 50 mL Schlenk tube, benzamide (1a or 13) (0.1 mmol), Pd(OTf)₂(MeCN)₄ (5.7 mg, 0.01 mmol), N-fluoro-2,4,6-trimethylpyridinium triflate (43.4 mg, 0.15 mmol), NMP (5.0 mg, 0.05 mmol) were dissolved in dry trifluorotoluene (1.0 mL) under N₂. The tube was sealed with a Teflon lined cap and the reaction mixture was stirred at 120° C. for 2 h. After cooling to room temperature, the mixture was concentrated in vacuo, and the residue was purified by column chromatography on silica gel with a gradient eluent of hexane and ethyl acetate to afford the product (1b or 13a, respectively). Following purification, a mixture of starting material and fluorinated products was obtained. In each case, the ratio (starting material/product) was determined by analysis of the NMR spectrum. Isolated yields were reported as averages of 5 runs.

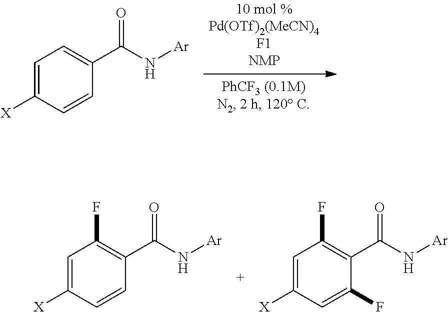

General Procedure C:

In a 50 mL Schlenk tube, benzamide (1, 4 or 8) (0.1 mmol), Pd(OTf)₂(MeCN)₄ (5.7 mg, 0.01 mmol), N-fluoro-2,4,6-trimethylpyridinium triflate (86.8 mg, 0.30 mmol), NMP (5.0 mg, 0.05 mmol) were dissolved in dry trifluorotoluene (1.0 mL) under N₂. The tube was sealed with a Teflon lined cap and the reaction mixture was stirred at 120° C. for 2 h. After cooling to room temperature, the mixture was concentrated in vacuo, and the residue was purified by column chromatography on silica gel with a gradient eluent of hexane and ethyl acetate to afford the product (1b, 4b or 8b, respectively). Following purification, a mixture of starting material and fluorinated products was obtained. In each case, the ratio (starting material/product) was determined by ¹H NMR spectrum analysis. Isolated yields were reported as averages of 5 runs.

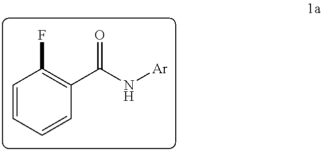

### 2-Fluoro-N-(2,3,5,6-tetrafluoro-4-(trifluoromethyl)phenyl)benzamide (1a)

Substrate 1 (0.1 mmol) was fluorinated following the general procedure A, with N-fluoro-2,4,6-trimethylpyridinium triflate (43.4 mg, 0.15 mmol) at 120° C. for 8 h. Further purification was carried out by preparative thin layer chromatography with an eluent of toluene:dichloromethane (1:1), affording 1a as a white solid (23.1 mg, 65%). Some di-fluorinated product 1b was isolated (2.7 mg, 7%), and some starting material was also recovered (3.4 mg, 10%).

The spectroscopic data for 1a was in agreement with the synthesized benzamide starting material 1a.

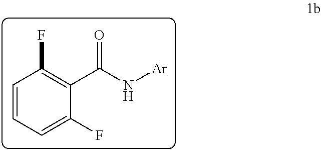

### 2,6-Difluoro-N-(2,3,5,6-tetrafluoro-4-(trifluoromethyl)phenyl)benzamide (1b)

Substrate 1a (0.1 mmol) was fluorinated following the general procedure B. After purification by column chromatography, 1b was obtained as a pale yellow solid (25.7 mg, 69%). Some starting material was recovered (3.4 mg, 10%).

¹H NMR (500 MHz, (CD₃)₂SO) δ 7.68 to 7.62 (m, 1H), 7.28 (t, J=8.1 Hz, 2H); ¹³C NMR (100 MHz, (CD₃)₂SO) δ 158.94 (dd, J=250.2, 7.2 Hz), 158.46, 133.09 (t, J=10.0 Hz), 113.45 (t, J=21.8 Hz), 112.20 (dd, J=19.4, 4.9 Hz); ¹⁹F NMR (375 MHz, (CD₃)₂SO) δ −55.00 (t, J=21.3 Hz, 3F), −113.01 (s, 2F), −141.74 to −142.09 (m, 4F); IR (neat) ν 3140, 2924, 1684, 1511, 1485, 1469, 1346, 1238, 1150, 1136, 996, 795 cm⁻¹; HRMS (ESI-TOF) Calcd for C₁₄H₅F₉NO (MH⁺): 374.0222. found: 374.0216.

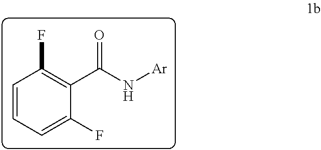

### 2,6-Difluoro-N-(2,3,5,6-tetrafluoro-4-(trifluoromethyl)phenyl)benzamide (1b)

Substrate 1 (0.1 mmol) was fluorinated following the general procedure C. After purification by column chromatography, 1b was obtained as a pale yellow solid (27.6 mg, 74%).

The spectroscopic data was in agreement with that obtained using general procedure B on substrate 1a.

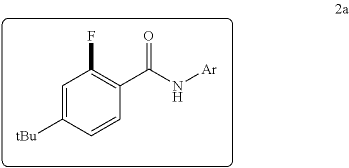

### 4-(tert-Butyl)-2-fluoro-N-(2,3,5,6-tetrafluoro-4-(trifluoromethyl)phenyl)benzamide (2a)

Substrate 2 (0.1 mmol) was fluorinated following the general procedure A, with N-fluoro-2,4,6-trimethylpyridinium triflate (43.4 mg, 0.15 mmol) at 120° C. for 2 h. After purification by column chromatography, 2a was obtained as a yellow solid (31.2 mg, 76%). A trace amount of di-fluorinated product 2b was isolated, and some starting material was also recovered (5.1 mg, 13%).

¹H NMR (600 MHz, CD₃OD) δ 7.80 (t, J=8.0 Hz, 1H), 7.41 (dd, J=8.2, 1.7 Hz, 1H), 7.33 (dd, J=13.2, 1.6 Hz, 1H), 1.37 (s, 9H); ¹³C NMR (150 MHz, (CD₃)₂SO) δ 162.50, 159.52 (d, J=250.6 Hz), 158.02 (d, J=7.3 Hz), 130.11 (d, J=2.4 Hz), 121.56 (d, J=2.7 Hz), 119.18 (d, J=13.8 Hz), 113.46 (d, J=22.2 Hz), 35.02, 30.64; ¹⁹F NMR (375 MHz, (CD₃)₂SO) δ −54.83 (t, J=21.0 Hz, 3F), −112.62 (s, 1F), −141.89 to −141.99 (m, 2F), −142.11 to −142.36 (m, 2F); IR (neat) ν 3386, 2978, 1682, 1510, 1469, 1458, 1341, 1235, 1140, 998 cm⁻¹; HRMS (ESI-TOF) Calcd for C₁₈H₁₄F₈NO (MH⁺): 412.0942. found: 412.0942.

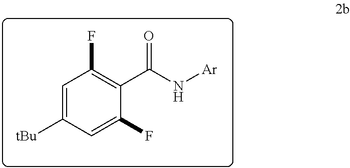

### 4-(tert-Butyl)-2,6-difluoro-N-(2,3,5,6-tetrafluoro-4-(trifluoromethyl)phenyl)benzamide (2b)

Substrate 2 (0.1 mmol) was fluorinated following the general procedure C. After purification by column chromatography, 2b was obtained as a yellow solid (37.7 mg, 88%).

¹H NMR (600 MHz, CDCl₃) δ 7.77 (br s, 1H), 7.04 (d, J=10.7 Hz, 2H), 1.33 (s, 9H); ¹³C NMR (100 MHz, CDCl₃) δ 160.70 (dd, J=253.8, 6.8 Hz), 159.57, 158.15, 109.88 (dd, J=23.3, 2.8 Hz), 35.69, 30.81; ¹⁹F NMR (375 MHz, CDCl₃) δ −56.63 (t, J=21.8 Hz, 3F), −111.09 (s, 2F), −140.98 to −141.19 (m, 2F), −142.48 to −142.53 (m, 2F); IR (neat) ν 3239, 2970, 1684, 1638, 1509, 1480, 1420, 1341, 1234, 1149, 999, 716 cm⁻¹; HRMS (ESI-TOF) Calcd for C₁₈H₁₃F₉NO (MH⁺): 430.0848. found: 430.0842.

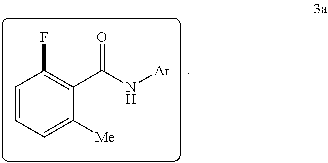

### 2-Fluoro-6-methyl-N-(2,3,5,6-tetrafluoro-4-(trifluoromethyl)phenyl)benzamide (3a)

Substrate 3 (0.1 mmol) was fluorinated following the general procedure A, with N-fluoro-2,4,6-trimethylpyridinium triflate (86.8 mg, 0.30 mmol) at 120° C. for 2 h. After purification by column chromatography, 3a was obtained as a white solid (28.8 mg, 78%).

¹H NMR (600 MHz, CD₃OD) δ 7.40 (dt, J=8.0, 6.0 Hz, 1H), 7.15 (d, J=7.7 Hz, 1H), 7.07 (t, J=8.8 Hz, 1H), 2.44 (s, 3H); ¹³C NMR (100 MHz, CD₃OD) δ 166.12, 160.59 (d, J=246.6 Hz), 139.39 (d, J=2.5 Hz), 132.56 (d, J=8.8 Hz), 127.27 (d, J=3.0 Hz), 125.13 (d, J=17.7 Hz), 114.05 (d, J=21.6 Hz), 19.00 (d, J=2.0 Hz); ¹⁹F NMR (375 MHz, (CD₃)₂SO) δ −56.64 (t, J=21.9 Hz, 3F), −117.53 (s, 1F), −142.84 to −143.12 (m, 2F), −143.41 to −143.45 (m, 2F); IR (neat) ν 3129, 2956, 1675, 1484, 1466, 1351, 1138, 996 cm⁻¹; HRMS (ESI-TOF) Calcd for C₁₅H₈F₈NO (MH⁺): 370.0473. found: 370.0470.

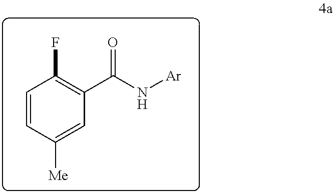

### 2-Fluoro-5-methyl-N-(2,3,5,6-tetrafluoro-4-(trifluoromethyl)phenyl)benzamide (4a)

Substrate 4 (0.1 mmol) was fluorinated following the general procedure A, with N-fluoro-2,4,6-trimethylpyridinium triflate (86.8 mg, 0.30 mmol) at 100° C. for 2 h. Further purification was carried out by preparative thin layer chromatography with an eluent of trifluorotoluene:dichloromethane (1:1), affording 4a as a white solid (27.8 mg, 75%).

¹H NMR (600 MHz, (CD₃)₂SO) δ 10.94 (s, 1H), 7.54 (dd, J=6.7, 1.7 Hz, 1H), 7.46 to 7.44 (m, 1H), 7.28 (dd, J=10.2, 8.6 Hz, 1H), 2.35 (s, 3H); ¹³C NMR (150 MHz, (CD₃)₂SO) δ 162.69, 157.63 (d, J=248.6 Hz), 134.14 (d, J=8.3 Hz), 134.05 (d, J=3.4 Hz), 130.31 (d, J=1.7 Hz), 121.74 (d, J=14.0 Hz), 116.22 (d, J=21.7 Hz), 19.97; ¹⁹F NMR (375 MHz, (CD₃)₂SO) δ −54.84 (t, J=20.8 Hz, 3F), −118.04 (s, 1F), −141.92 to −142.26 (m, 4F); IR (neat) ν 3261, 1687, 1478, 1351, 1242, 1139, 996 cm⁻¹; HRMS (ESI-TOF) Calcd for C₁₅H₈F₈NO (MH⁺): 370.0473. found: 370.0479.

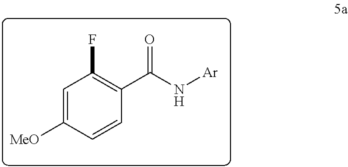

### 2-Fluoro-4-methoxy-N-(2,3,5,6-tetrafluoro-4-(trifluoromethyl)phenyl)benzamide (5a)

Substrate 5 (0.1 mmol) was fluorinated following the general procedure A, with N-fluoro-2,4,6-trimethylpyridinium triflate (86.8 mg, 0.30 mmol) at 100° C. for 24 h. After purification by column chromatography, 5a was obtained as a white solid (20.8 mg, 54%). A trace amount of di-fluorinated product was observed by ¹H NMR, and some starting material was also recovered (7.3 mg, 20%).

¹H NMR (600 MHz, (CD₃)₂SO)S 10.66 (s, 1H), 7.73 (t, J=8.6 Hz, 1H), 7.01 (dd, J=12.8, 2.3 Hz, 1H), 6.93 (dd, J=8.7, 2.4 Hz, 1H), 3.86 (s, 3H); ¹³C NMR (150 MHz, (CD3)₂SO) δ 163.71 (d, J=11.5 Hz), 162.11 (d, J=1.5 Hz), 161.14 (d, J=251.9 Hz), 131.84 (d, J=3.8 Hz), 113.68 (d, J=13.1 Hz), 110.89 (d, J=2.5 Hz), 102.12 (d, J=25.8 Hz), 56.14; ¹⁹F NMR (375 MHz, (CD₃)₂SO) δ −54.83 (t, J=21.1 Hz, 3F), −109.02 (s, 1F), −141.95 to 142.05 (m, 2F), −142.23 to −142.49 (m, 2F); IR (neat) ν 3363, 3014, 1677, 1620, 1514, 1503, 1456, 1442, 1339, 1232, 1194, 1162, 995, 955 cm⁻¹; HRMS (ESI-TOF) Calcd for C₁₅H₈F₈NO₂ (MH⁺): 386.0422. found: 386.0416.

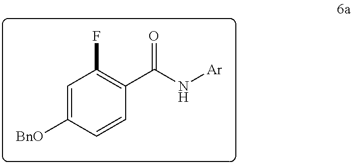

### 4-(Benzyloxy)-2-fluoro-N-(2,3,5,6-tetrafluoro-4-(trifluoromethyl)phenyl)benzamide (6a)

Substrate 6 (0.1 mmol) was fluorinated following the general procedure A, with N-fluoro-2,4,6-trimethylpyridinium triflate (86.8 mg, 0.30 mmol) at 120° C. for 3 h. After purification by column chromatography, 6a was obtained as a white solid (36.0 mg, 78%). Some starting material was also recovered (5.3 mg, 12%).

¹H NMR (600 MHz, (CD₃)₂SO) δ 10.69 (s, 1H), 7.73 (t, J=8.6 Hz, 1H), 7.47 (d, J=7.2 Hz, 2H), 7.41 (t, J=7.5 Hz, 2H), 7.36 (t, J=7.3 Hz, 1H), 7.10 (dd, J=12.8, 2.3 Hz, 1H), 7.01 (dd, J=8.7, 2.3 Hz, 1H), 5.23 (s, 2H); ¹³C NMR (150 MHz, (CD₃)₂SO) δ 162.61 (d, J=11.6 Hz), 162.10, 161.01 (d, J=251.9 Hz), 136.12, 131.80, 128.57, 128.18, 127.92, 113.94 (d, J=13.1 Hz), 111.64 (d, J=2.4 Hz), 102.99 (d, J=25.8 Hz), 70.02; ¹⁹F NMR (375 MHz, (CD₃)₂SO) δ −54.75 (t, J=21.0 Hz, 3F), −109.03 (s, 1F), −141.92 to −142.02 (m, 2F), −142.14 to −142.41 (m, 2F); IR (neat) ν 3435, 2921, 1690, 1624, 1512, 1471, 1342, 1233, 1224, 1135, 994 cm⁻¹; HRMS (ESI-TOF) Calcd for C₂₁H₁₂F₈NO₂ (MH⁺): 462.0735. found: 462.0747.

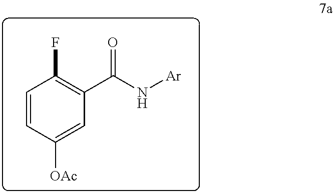

### 4-Fluoro-3-((2,3,5,6-tetrafluoro-4-(trifluoromethyl)phenyl)carbamoyl)phenyl acetate (7a)

Substrate 7 (0.1 mmol) was fluorinated following the general procedure A, with N-fluoro-2,4,6-trimethylpyridinium triflate (86.8 mg; 0.30 mmol) at 120° C. for 24 h. After purification by column chromatography, 7a was obtained as a white solid (15.3 mg, 37%). ¹H NMR (600 MHz, (CD₃)₂SO) δ 7.52 (dd, J=5.7, 2.7 Hz, 1H), 7.47 (t, J=9.2 Hz, 1H), 7.45 to 7.42 (m, 1H), 2.29 (s, 3H); ¹³C NMR (150 MHz, (CD₃)₂SO) δ 169.27, 161.78, 156.73 (d, J=249.3 Hz), 146.39 (d, J=2.6 Hz), 127.29 (d, J=9.0 Hz), 123.44 (d, J=2.6 Hz), 122.89 (d, J=16.3 Hz), 117.60 (d, J=24.1 Hz), 20.78; ¹⁹F NMR (375 MHz, (CD₃)₂SO) δ −54.81 (t, J=21.3 Hz, 3F), −116.64 (s, 1F), −141.90 to −142.06 (m, 4F); IR (neat) ν 3266, 2924, 1760, 1685, 1513, 1477, 1351, 1216, 1187, 1139, 998 cm⁻¹; HRMS (ESI-TOF) Calcd for C₁₆H₈F₈NO₃ (MH⁺): 414.0371. found: 414.0363.

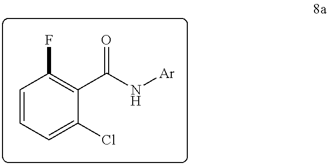

### 2-Chloro-6-fluoro-N-(2,3,5,6-tetrafluoro-4-(trifluoromethyl)phenyl)benzamide (8a)

Substrate 8 (0.1 mmol) was fluorinated following the general procedure A, with N-fluoro-2,4,6-trimethylpyridinium triflate (86.8 mg, 0.30 mmol) at 120° C. for 12 h. After purification by column chromatography, 8a was obtained as a pale yellow solid (26.5 mg, 68%). Some starting material was also recovered (8.9 mg, 24%).

¹H NMR (600 MHz, (CD₃)₂SO) δ 7.61 (dd, J=14.6, 8.3 Hz, 1H), 7.50 (d, J=8.1 Hz, 1H), 7.43 (t, J=8.6 Hz, 1H); ¹³C NMR (150 MHz, (CD₃)₂SO) δ 160.29, 158.77 (d, J=249.2 Hz), 132.64 (d, J=9.1 Hz), 130.97 (d, J=5.4 Hz), 125.78 (d, J=3.1 Hz), 124.20 (d, J=22.2 Hz), 114.98 (d, J=21.1 Hz); ¹⁹F NMR (375 MHz, (CD₃)₂SO) δ −54.97 (t, J=21.1 Hz, 3F), −113.30 (s, 1F), −141.82 to −141.97 (m, 4F); IR (neat) ν 3138, 2962, 1681, 1511, 1484, 1451, 1350, 1243, 1153, 1135, 997, 908, 789 cm⁻¹; HRMS (ESI-TOF) Calcd for C₁₄H₅ClF₈NO (MH⁺): 389.9926. found: 389.9932.

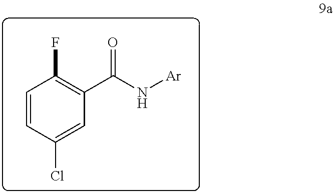

### 5-Chloro-2-fluoro-N-(2,3,5,6-tetrafluoro-4-(trifluoromethyl)phenyl)benzamide (9a)

Substrate 9 (0.1 mmol) was fluorinated following the general procedure A, with N-fluoro-2,4,6-trimethylpyridinium triflate (86.8 mg, 0.30 mmol) at 120° C. for 24 h. Further purification was carried out by preparative thin layer chromatography with an eluent of toluene:dichloromethane (4:1), affording 9a as a pale yellow solid (28.4 mg, 73%).

¹H NMR (400 MHz, (CD₃)₂SO) δ 7.78 (dd, J=5.9, 2.7 Hz, 1H), 7.75 to 7.71 (m, 1H), 7.48 (dd, J=9.7, 9.0 Hz, 1H); ¹³C NMR (125 MHz, (CD₃)₂SO) δ 161.20, 158.08 (d, J=251.9 Hz), 133.42 (d, J=8.8 Hz), 129.63 (d, J=2.4 Hz), 128.51 (d, J=3.2 Hz), 123.80 (d, J=15.9 Hz), 118.60 (d, J=23.8 Hz); ¹⁹F NMR (375 MHz, (CD₃)₂SO) δ −54.86 (t, J=20.9 Hz, 3F), −115.21 (s, 1F), −141.78 to −142.14 (m, 4F); IR (neat) ν 3252, 1684, 1512, 1478, 1351, 1241, 1145, 999 cm⁻¹; HRMS (ESI-TOF) Calcd for C₁₄H₅ClF₈NO (MH⁺): 389.9926. found: 389.9935.

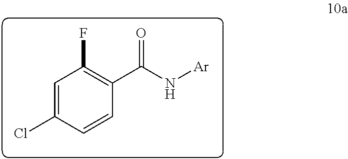

### 4-Chloro-2-fluoro-N-(2,3,5,6-tetrafluoro-4-(trifluoromethyl)phenyl)benzamide (10a)

Substrate 10 (0.1 mmol) was fluorinated following the general procedure A, with N-fluoro-2,4,6-trimethylpyridinium triflate (86.8 mg, 0.30 mmol) at 120° C. for 24 h. Further purification was carried out by preparative thin layer chromatography with an eluent of toluene:dichloromethane (1:1), affording 10a as a pale yellow solid (25.7 mg, 66%). Some di-fluorinated product 10b was isolated (3.3 mg, 8%), and some starting material was also recovered (2.6 mg, 7%).

¹H NMR (600 MHz, (CD₃)₂SO) δ 7.77 (t, J=8.0 Hz, 1H), 7.67 (dd, J=10.1, 1.5 Hz, 1H), 7.47 (dd, J=8.3, 1.5 Hz, 1H); ¹³C NMR (150 MHz, (CD₃)₂SO) δ 161.75, 159.49 (d, J=255.2 Hz), 137.49 (d, J=10.6 Hz), 131.73 (d, J=2.9 Hz), 125.17 (d, J=3.4 Hz), 121.20 (d, J=14.1 Hz), 117.18 (d, J=25.7 Hz); ¹⁹F NMR (375 MHz, (CD₃)₂SO) δ −54.89 (t, J=20.8 Hz, 3F), −109.87 (s, 1F), −141.91 to −142.18 (m, 4F); IR (neat) ν 3255, 1677, 1609, 1530, 1509, 1471, 1338, 1150, 998, 919 cm⁻¹; HRMS (ESI-TOF) Calcd for C₁₄H₅ClF₈NO (MH⁺): 389.9926. found: 389.9915.

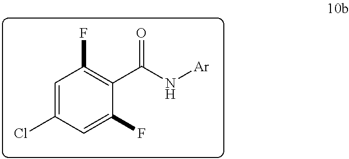

### 4-Chloro-2,6-difluoro-N-(2,3,5,6-tetrafluoro-4-(trifluoromethyl)phenyl)benzamide (10b)

Substrate 10 (0.1 mmol) was fluorinated following the general procedure C. After purification by column chromatography, 10b was obtained as a pale yellow solid (26.9 mg, 66%). Some mono-fluorinated product 10a was also isolated (2.7 mg, 7%).

¹H NMR (600 MHz, (CD₃)₂SO)S 7.61 (d, J=7.5 Hz, 2H); ¹³C NMR (150 MHz, (CD3)₂SO) δ 158.96 (dd, J=252.7, 8.9 Hz), 157.63, 136.68 (t, J=13.3 Hz), 113.48 (dd, J=24.2, 4.6 Hz), 112.63 (t, J=21.8 Hz); ¹⁹F NMR (375 MHz, (CD₃)₂SO) δ −54.88 (t, J=21.1 Hz, 3F), −110.89 (s, 2F), −141.63 to −142.08 (m, 4F); IR (neat) ν 3222, 2925, 1700, 1622, 1509, 1482, 1340, 1235, 1151, 997 cm⁻¹; HRMS (ESI-TOF) Calcd for C₁₄H₄ClF₉NO (MH⁺): 407.9832. found: 407.9844.

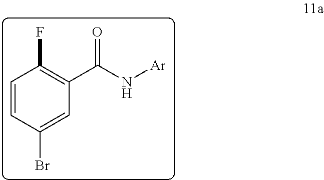

### 3-Bromo-2-fluoro-N-(2,3,5,6-tetrafluoro-4-(trifluoromethyl)phenyl)benzamide (11a)

Substrate 11 (0.1 mmol) was fluorinated following the general procedure A, with N-fluoro-2,4,6-trimethylpyridinium triflate (86.8 mg, 0.30 mmol) at 120° C. for 12 h. Further purification was carried out by preparative thin layer chromatography with an eluent of trifluorotoluene:dichloromethane (1:1), affording 11a as a pale yellow solid (33.9 mg, 78%).

¹H NMR (600 MHz, (CD₃)₂SO) δ 7.89 (dd, J=6.1, 2.6 Hz, 1H), 7.86 to 7.84 (m, 1H), 7.41 (t, J=9.1 Hz, 1H); ¹³C NMR (150 MHz, (CD₃)₂SO) δ 161.15, 158.66 (d, J=252.4 Hz), 136.42 (d, J=8.7 Hz), 132.55 (d, J=2.1 Hz), 124.23 (d, J=15.5 Hz), 119.01 (d, J=23.4 Hz), 116.26 (d, J=3.2 Hz); ¹⁹F NMR (375 MHz, (CD₃)₂SO) δ −55.92 (t, J=21.8 Hz, 3F), −115.07 (s, 1F), −142.16 to −142.44 (m, 2F), −142.60 to −142.70 (m, 2F); IR (neat) ν 3248, 1681, 1510, 1477, 1347, 1241, 1143, 998, 824 cm⁻¹; HRMS (ESI-TOF) Calcd for C₁₄H₅BrF₈NO (MH⁺): 433.9421. found: 433.9426.

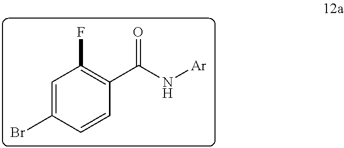

### 4-Bromo-2-fluoro-N-(2,3,5,6-tetrafluoro-4-(trifluoromethyl)phenyl)benzamide (12a)

Substrate 12(0.1 mmol) was fluorinated following the general procedure A, with N-fluoro-2,4,6-trimethylpyridinium triflate (86.8 mg, 0.30 mmol) at 120° C. for 12 h. After purification by column chromatography, 12a was obtained as a pale yellow solid (30.0 mg, 69%). Some di-fluorinated product was observed by ¹H NMR (9%).

¹H NMR (600 MHz, (CD₃)₂SO) δ 7.81 (dd, J=9.9, 1.7 Hz, 1H), 7.69 (t, J=7.9 Hz, 1H), 7.61 (dd, J=8.3, 1.7 Hz, 1H); ¹³C NMR (150 MHz, (CD₃)₂SO) δ 161.87, 159.30 (d, J=256.1 Hz), 131.83 (d, J=2.7 Hz), 128.09 (d, J=3.4 Hz), 125.80 (d, J=9.7 Hz), 121.54 (d, J=14.1 Hz), 119.96 (d, J=25.2 Hz); ¹⁹F NMR (375 MHz, (CD₃)₂SO) δ −55.93 (t, J=21.9 Hz, 3F), −110.32 (s, IF), −142.26 to −142.55 (m, 2F), −142.82 to −142.92 (m, 2F); IR (neat) ν 3250, 2924, 1681, 1605, 1510, 1485, 1472, 1342, 1241, 1153, 997, 911 cm⁻¹; HRMS (ESI-TOF) Calcd for C₁₄H₅BrF₈NO (MH⁺): 433.9421. found: 433.9415.

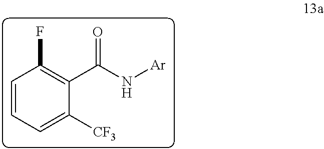

### 2-Fluoro-N-(2,3,5,6-tetrafluoro-4-(trifluoromethyl)phenyl)-6-(trifluoromethyl)benzamide (13a)

Substrate 13 (0.1 mmol) was fluorinated following the general procedure B. Further purification was carried out by preparative thin layer chromatography with an eluent of trifluorotoluene:dichloromethane (1:1), affording 13a as a yellow solid (29.6 mg, 70%). ¹H NMR (600 MHz, (CD₃)₂SO) δ 7.84 to 7.76 (m, 3H); ¹³C NMR (150 MHz, (CD₃)₂SO) δ 160.60, 158.57 (d, J=247.5 Hz), 132.84 (d, J=8.7 Hz), 127.87 (dq, J=32.2, 3.6 Hz), 122.85 (dq, J=274.0, 3.0 Hz), 122.64, 120.59 (d, J=21.6 Hz); ¹⁹F NMR (375 MHz, (CD₃)₂SO) δ −54.88 (t, J=21.2 Hz, 3F), −57.79 (s, 3F), −113.88 (s, 1F), −141.67 to −141.88 (m, 2F), −142.45 to −142.49 (m, 2F); IR (neat) ν 3217, 2924, 1696, 1535, 1509, 1487, 1475, 1341, 1330, 1236, 1143, 998, 916 cm⁻¹; HRMS (ESI-TOF) Calcd for C₁₅H₅F₁₁NO (MH⁺): 424.0190. found: 424.0202.

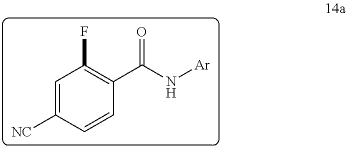

### 4-Cyano-2-fluoro-N-(2,3,5,6-tetrafluoro-4-(trifluoromethyl)phenyl)benzamide (14a)

Substrate 14 (0.1 mmol) was fluorinated following the general procedure A, with N-fluoro-2,4,6-trimethylpyridinium triflate (86.8 mg, 0.30 mmol) at 120° C. for 24 h. Further purification was carried out by preparative thin layer chromatography with an eluent of toluene:dichloromethane (1:1), affording 14a as a pale yellow solid (23.6 mg, 62%). Some starting material was also recovered (5.1 mg, 14%).

¹H NMR (600 MHz, (CD₃)₂SO) δ 8.10 (d, J=9.5 Hz, 1H), 7.92 to 7.87 (m, 2H); ¹³C NMR (150 MHz, (CD₃)₂SO) δ 161.45, 158.69 (d, J=253.4 Hz), 131.42 (d, J=2.7 Hz), 129.05 (d, J=3.7 Hz), 127.10 (d, J=14.9 Hz), 120.70 (d, J=25.9 Hz), 117.07 (d, J=2.2 Hz), 115.52 (d, J=10.0 Hz); ¹⁹F NMR (375 MHz, (CD₃)₂SO) δ −54.90 (t, J=21.4 Hz, 3F), −110.88 (s, 1F), −141.83 to −141.98 (m, 4F); IR (neat) ν 3251, 2243, 1685, 1510, 1499, 1477, 1349, 1159, 995 cm⁻¹; HRMS (ESI-TOF) Calcd for C₁₅H₅F₈N₂O (MH⁺): 381.0269. found: 381.0266.

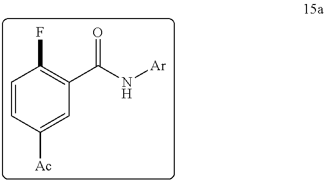

### 5-Acetyl-2-fluoro-N-(2,3,5,6-tetrafluoro-4-(trifluoromethyl)phenyl)benzamide (15a)

Substrate 15 (0.1 mmol) was fluorinated following the general procedure A, with N-fluoro-2,4,6-trimethylpyridinium triflate (86.8 mg, 0.30 mmol) at 120° C. for 24 h. Further purification was carried out by preparative thin layer chromatography with an eluent of toluene:dichloromethane (1:1), affording 15a as a white solid (14.3 mg, 36%). Some starting material was also recovered (8.7 mg, 23%).

¹H NMR (600 MHz, CD₃OD) δ 8.44 (dd, J=6.8, 2.2 Hz, 1H), 8.27 (ddd, J=8.5, 4.8, 2.3 Hz, 1H), 7.44 (dd, J=9.8, 9.0 Hz, 1H), 2.65 (s, 3H); ¹³C NMR (150 MHz, CD₃OD) δ 197.77, 164.23 (d, J=259.4 Hz), 164.07, 135.39 (d, J=10.2 Hz), 135.20 (d, J=3.2 Hz), 132.53 (d, J=3.5 Hz), 123.30 (d, J=14.6 Hz), 118.15 (d, J=23.3 Hz), 26.66; ¹⁹F NMR (375 MHz, CD₃OD) δ −55.92 (t, J=21.9 Hz, 3F), −106.39 (s, 1F), −142.28 to −142.56 (m, 2F), −142.83 to −142.93 (m, 2F); IR (neat) ν 3272, 2930, 1689, 1510, 1474, 1340, 1225, 1239, 1147, 1000 cm⁻¹; HRMS (ESI-TOF) Calcd C₁₆H₆F₈NO₂ ([M−H]⁻): 396.0276. found: 396.0283.

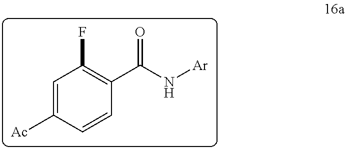

### 4-Acetyl-2-fluoro-N-(2,3,5,6-tetrafluoro-4-(trifluoromethyl)phenyl)benzamide (16a)

Substrate 16 (0.1 mmol) was fluorinated following the general procedure A, with N-fluoro-2,4,6-trimethylpyridinium triflate (86.8 mg, 0.30 mmol) at 120° C. for 24 h. After purification by column chromatography, 16a was obtained as a white solid (17.5 mg, 44%). Some starting material was also recovered (3.0 mg, 8%).

¹H NMR (600 MHz, (CD₃)₂SO) δ 7.93 to 7.90 (m, 2H), 7.88 to 7.86 (m, 1H), 2.66 (s, 3H); ¹³C NMR (150 MHz, (CD₃)₂SO) δ 196.71, 162.05, 159.25 (d, J=252.3 Hz), 141.04 (d, J=6.5 Hz), 130.77 (d, J=2.0 Hz), 126.18 (d, J=15.0 Hz), 124.30 (d, J=3.2 Hz), 115.90 (d, J=22.8 Hz), 27.11; ¹⁹F NMR (375 MHz, CD₃OD) δ −55.94 (t, J=21.9 Hz, 3F), −112.35 (s, 1F), −142.19 to −142.45 (m, 2F), −142.77 to −142.85 (m, 2F); IR (neat) ν 3255, 2925, 1682, 1511, 1494, 1476, 1350, 1152, 996 cm⁻¹; HRMS (ESI-TOF) Calcd for C₁₆H₈F₈NO₂ (MH⁺): 398.0422. found: 398.0425.

### Example 6

### Benzamide hydrolysis

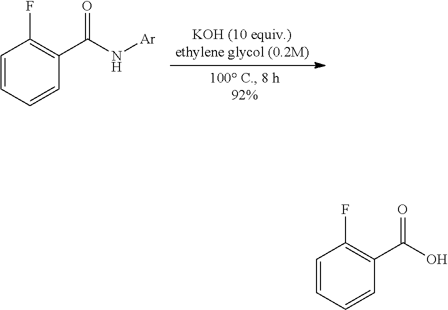

KOH (112.2 mg, 2.0 mmol) was added to a solution of 1a (71.0 mg, 0.2 mmol) in ethylene glycol (1 mL). The mixture was heated to 100° C. and stirred for 8 h. After being cooled to room temperature, dichloromethane (20 mL) was added. The whole mixture was washed three times with 3 N HCl, then once with brine. The organic phase was dried over Na₂SO₄ and concentrated in vacuo to give the carboxylic acid (25.9 mg, 92%). The ¹H NMR spectrum was in agreement with that obtained using commercial 2-fluorobenzoic acid.

Although the subject matter has been described in terms of exemplary embodiments, it is not limited thereto. Rather, the appended claims should be construed broadly, to include equivalent alterations, modifications, other variants and embodiments, which may be made by those skilled in the art. In addition, while a particular feature of the invention may have been disclosed with respect to only one of several implementations, such feature may be combined with one or more other features of the other implementations as may be desired and advantageous for any given or particular application.

The Abstract of the disclosure will allow the reader to quickly ascertain the nature of the technical disclosure. It is submitted with the understanding that it will not be used to interpret or limit the scope or meaning of the following claims.

What is claimed is:

1. A method of fluorinating a benzoic acid or derivative compound comprising:
reacting a benzoic acid or derivative compound having the formula: 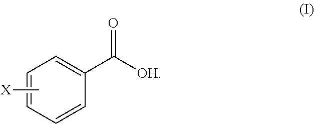
to form a benzamide compound having the formula: 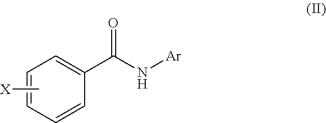
wherein X is at least one substitution group attached with a benzene ring and is independently selected from the group of radicals consisting of H, halo, C₁₋₂₀ alkyl, C₂₋₂₀ alkenyl, C₂₋₂₀ alkynyl, C₁₋₂₀ alkoxyl, C₆₋₂₀ aryl, C₇₋₂₀ alkylaryl, C₄₋₂₀ heterocycle, C₄₋₂₀ heteroaryl, C₄₋₂₀ alkyl heterocycle, C₇₋₂₀ alkyl heteroaryl, C₆₋₂₀ aryloxy, —OH, —CO, —COOH, —NO₂, —CN, —NH₂, —N₃, —CF₃, —OCF₃, —NR³R³′, —N(O)R³, —SH, —SR³, —SOR³, —SO₂R³, —C(O)R³, —CO₂R³, —C(O)NR₃R³′, and —OC(O)R³; wherein the alkyl, alkenyl, alkynyl, alkoxy, aryl, heterocycle, heteroaryl, or aryloxy may be substituted or unsubstituted and wherein in the alkyl portion one or more —CH₂—, CH₂CH₂—, —(CH₂)ₙ— groups are each optionally replaced by —O— or —NH—; wherein n is an integer equal to or greater than 1;
R³ and R³′ are each independently selected from the group of radicals consisting of H, C₁₋₂₀ alkyl, C₂₋₂₀ alkenyl, C₂₋₂₀ alkynyl, C₆₋₂₀ aryl; C₇₋₂₀ alkylaryl, C₄₋₂₀ heterocycle, C₄₋₂₀ heteroaryl, C₄₋₂₀ alkylheterocycle, and C₇₋₂₀ alkylheteroaryl;
wherein the alkyl, alkenyl, alkynyl, aryl, heterocycle, or heteroaryl, be substituted or unsubstituted and wherein in the alkyl portion one or more —CH₂—, CH₂CH₂—, —(CH₂)ₙ— groups are each optionally replaced by —O— or —NH—; n is an integer equal to or greater than 1;
wherein Ar is an aromatic substitution group comprising at least one fluorine atom;
and
reacting the benzamide compound having the formula (II) and a mixture comprising:
a palladium (H) catalyst; and
a fluorinating reagent;
to form at least one of the ortho-fluorinated compounds having the formulae 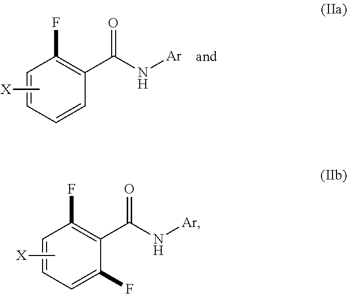
wherein X and Ar are described above.

2. The method of claim 1 further comprising:
transforming the at least one of the ortho-fluorinated compounds having the formula (IIa) and (IIb) into an ortho-fluorinated benzoic acid or derivative compound having the formula: 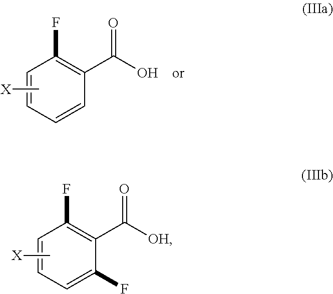
wherein X is described above in claim 1.

3. The method of claim 1, wherein X is one substitution group independently selected from the group of radicals consisting of H, halo, C₁₋₆ alkyl, C₂₋₆ alkenyl, C₂₋₆ alkynyl, C₁₋₆ alkoxyl, C₆₋₂₀ aryl; C₇₋₂₀ alkylaryl, C₄₋₂₀ heterocycle, C₄₋₂₀ heteroaryl, C₄₋₂₀ alkylheterocycle, and C₇₋₂₀ alkylheteroaryl; —OH, —CO, —COOH, —NO₂, —CN, —NH₂, —N₃, —CF₃, —OCF₃, —NR³R³′, —N(O)R³, —SH, —SR³, —SOR³, —SO₂R³, —C(O)R³, —CO₂R³, —C(O)NR₃R³′, and —OC(O)R³; wherein the alkyl, alkenyl, alkynyl, alkoxy, aryl, heterocycle, heteroaryl, or aryloxy may be substituted or unsubstituted and wherein in the alkyl portion one or more: —CH₂—, CH₂CH₂—, —(CH₂)ₙ— groups are each optionally replaced by —O— or —NH—; wherein n is an integer equal to or greater than 1.

4. The method of claim 1, wherein at least two of the at least one X group are joined together to form a bicyclic or tricyclic ring structure with the benzene ring to which at least two of the at least one X group are attached, the bicyclic or tricyclic ring structure selected from the group consisting of alkyl, aryl, heterocycle and heteroaryl ring structures.

5. The method of claim 1, wherein Ar is an aryl group comprising at least two fluorine substitution groups.

6. The method of claim 5, wherein Ar is a 2,3,5,6-tetrafluoro-4-(trifluoromethyl) phenyl group.

7. The method of claim 5, wherein Ar is a 2,3,5,6-tetrafluoro-4-cyano phenyl group.

8. The method of claim 1, wherein the palladium (II) catalyst is Pd (OTs)₂(CH₃CN)₄, Pd(OTf)₂(CH₃CN)₄, Pd(OTf)₂(H₂O)₄, Pd(NTf₂)₂, Pd(OTf)₂ or Pd(TFA)₂, wherein Ts is toluenesulfonyl, Tf is trifluoromethanesulfonyl and TFA is trifluoroacetate.

9. The method of claim 8, wherein the palladium (II) catalyst is Pd(OTf)₂(CH₃CN)₄.

10. The method of claim 1, wherein 5-20 mol % of the palladium (II) catalyst is used.

11. The method of claim 1, wherein the fluorinating agent is a compound having the formula: 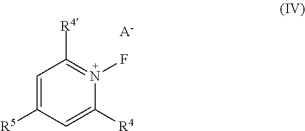
wherein A⁻ is a counter ion; and R⁴, R⁴′, and R⁵ are each independently hydrogen, halogen, C₁₋₁₂ alkyl, or C₂₋₁₂ alkenyl, wherein the alkyl or alkenyl may be substituted with one or more halogen.

12. The method of claim 11, wherein A⁻ is OTf BF₄⁻, or PF₆⁻.

13. The method of claim 11, wherein the fluorinating agent is a compound having the formula: 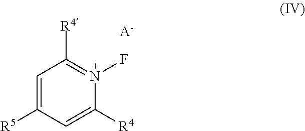
wherein
R⁴ and R⁴′ are each independently halogen, C₁₋₆ alkyl;
R⁵ is H or a C₁₋₆ alkyl; and
A⁻ is OTf⁻, BF₄⁻, or PF₆⁻.

14. The method of claim 13, wherein the fluorinating reagent is N-fluoro-2-4-6-trimethylpyridinium triflate.

15. The method of claim 1, wherein 1-5 equivalents of the fluorinating reagent is used.

16. The method of claim 1, wherein the mixture comprising a palladium (II) catalyst and a fluorinating reagent further comprises an additive and a solvent.

17. The method of claim 16, wherein the additive is N-Methyl-2-pyrrolidone (NMP).

18. The method of claim 16, wherein the solvent is trifluoromethyl benzene (PhCF₃), ethyl acetate (EtOAc) or CH₃CN.

19. The method of claim 16, wherein the solvent is CH₃CN.

20. The method of claim 1, wherein
Ar is a 2,3,5,6-tetrafluoro-4-(trifluoromethyl)phenyl group;
the palladium (II) catalyst is Pd(OTf)₂(CH₃CN)₄;
the fluorinating reagent is N-fluoro-2-4-6-trimethylpyridinium triflate;
the additive is NMP; and
the solvent is CH₃CN.

21. The method of claim 1, wherein reacting the benzamide compound having the formula (II) to form at least one of the ortho-fluorinated compounds having the formulae (IIa) and (IIb) is performed at a temperature under a heating condition.

22. The method of claim 21, the temperature is in the range of 80-120° C.

23. The method of claim 1, wherein the yield of monofluorinated product having the formula (IIa) is at least 50%, and the yield of difluorinated product having the formula (IIb) is less than 10%.

24. The method of claim 23, wherein the yield of monofluorinated product having the formula (IIa) is at least 80%, and the yield of difluorinated product having the formula (IIb) is less than 5%.

25. The method of claim 1, wherein the yield of difluorinated product having the formula (IIb) is at least 50%, and the yield of monofluorinated product having the formula (IIa) is less than 10%.

26. The method of claim 25, wherein the yield of difluorinated product having the formula (IIb) is at least 80%, and the yield of monofluorinated product having the formula (IIa) is less than 5%.

27. The method of claim 25, wherein the solvent is PhCF3.

28. A method of fluorinating a benzoic acid or derivative compound comprising:
reacting a compound having the formula: 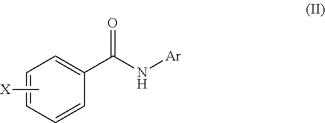
wherein X is at least one substitution group attached with a benzene ring and is independently selected from the group of radicals consisting of H, halo, C₁₋₂₀ alkyl, C₂₋₂₀ alkenyl, C₂₋₂₀ alkynyl, C₁₋₂₀ alkoxyl, C₆₋₂₀ aryl, C₇₋₂₀ alkylaryl, C₄₋₂₀ heterocycle, C₄₋₂₀ heteroaryl, C₄₋₂₀ alkyl heterocycle, C₇₋₂₀ alkyl heteroaryl, C₆₋₂₀ aryloxy, —OH, —CO, —COOH, —NO₂, —CN, —NH₂, —N₃, —CF₃, —NR³R³′, —N(O)R³, —SH, —SR³, —SOR³, —SO₂R³, —C(O)R³, —CO₂R³, —C(O)NR₃R³′, and —OC(O)R³; wherein the alkyl, alkenyl, alkynyl, alkoxy, aryl, heterocycle, heteroaryl, or aryloxy may be substituted or unsubstituted and wherein in the alkyl portion one or more —CH₂—, CH₂CH₂—, —(CH₂)ₙ— groups are each optionally replaced by —O— or —NH—; wherein n is an integer equal to or greater than 1;
R³ and R³′ are each independently selected from the group of radicals consisting of H, C₁₋₂₀ alkyl, C₂₋₂₀ alkenyl, C₂₋₂₀ alkynyl, C₆₋₂₀ aryl; C₇₋₂₀ alkylaryl, C₄₋₂₀ heterocycle, C₄₋₂₀ heteroaryl, C₄₋₂₀ alkylheterocycle, and C₇₋₂₀ alkylheteroaryl;
wherein the alkyl, alkenyl, alkynyl, aryl, heterocycle, or heteroaryl, be substituted or unsubstituted and wherein in the alkyl portion one or more —CH₂—, CH₂CH₂—, —(CH₂)ₙ— groups are each optionally replaced by —O— or —NH—; n is an integer equal to or greater than 1;
wherein Ar is an aromatic substitution group comprising at least one fluorine atom;
and a mixture comprising:
a palladium (II) catalyst; and
a fluorinating reagent;
to form at least one of the ortho-fluorinated compounds having the formulae 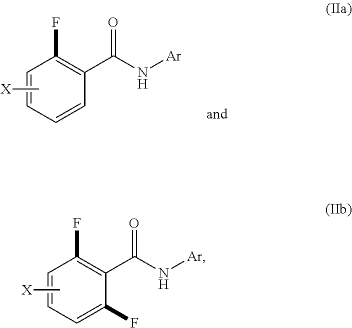
wherein X and Ar are described above.

29. The method of claim 28, wherein X is one substitution group independently selected from the group of radicals consisting of H, halo, C₁₋₆ alkyl, C₂₋₆ alkenyl, C₂₋₆ alkynyl, C₁₋₆ alkoxyl, C₆₋₂₀ aryl; C₇₋₂₀ alkylaryl, C₄₋₂₀ heterocycle, C₄₋₂₀ heteroaryl, C₄₋₂₀ alkylheterocycle, and C₇₋₂₀ alkylheteroaryl; —OH, —CO, —COOH, —NO₂, —CN, —NH₂, —N₃, —CF₃, —OCF₃, —NR³R³′, —N(O)R³, —SH, —SR³, —SOR³, —SO₂R³, —C(O)R³, —CO₂R³, —C(O)NR₃R³′, and —OC(O)R³; wherein the alkyl, alkenyl, alkynyl, alkoxy, aryl, heterocycle, heteroaryl, or aryloxy may be substituted or unsubstituted and wherein in the alkyl portion one or more: —CH₂—, CH₂CH₂—, —(CH₂)ₙ— groups are each optionally replaced by —O— or —NH—; wherein n is an integer equal to or greater than 1.

30. The method of claim 28, wherein at least two of the at least one X group are joined together to form a bicyclic or tricyclic ring structure with the benzene ring to which at least two of the at least one X group are attached, the bicyclic or tricyclic ring structure selected from the group consisting of alkyl, aryl, heterocycle and heteroaryl ring structures.

31. The method of claim 28, wherein Ar is an aryl group comprising at least two fluorine substitution groups.

32. The method of claim 31, wherein Ar is a 2,3,5,6-tetrafluoro-4-(trifluoromethyl) phenyl group.

33. The method of claim 31, wherein Ar is a 2,3,5,6-tetrafluoro-4-cyano phenyl group.

34. The method of claim 28, wherein the palladium (II) catalyst is Pd (OTs)₂(CH₃CN)₄, Pd(OTf)₂(CH₃CN)₄, Pd(OTf)₂(H₂O)₄, Pd(NTf₂)₂, Pd(OTf)₂ or Pd(TFA)₂, wherein Ts is toluenesulfonyl, Tf is trifluoromethanesulfonyl and TFA is trifluoroacetate.

35. The method of claim 34, wherein the palladium (II) catalyst is Pd(OTf)₂(CH₃CN)₄.

36. The method of claim 28, wherein 5-20 mol % of the palladium (II) catalyst is used.

37. The method of claim 28, wherein the fluorinating agent is a compound having the formula: 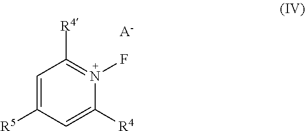
wherein A⁻ is a counter ion; and R⁴, R⁴′, and R⁵ are each independently hydrogen, halogen, C₁₋₁₂ alkyl, or C₂₋₁₂ alkenyl, wherein the alkyl or alkenyl may be substituted with one or more halogen.

38. The method of claim 37, wherein A⁻ is OTf⁻, BF₄⁻, or PF₆⁻.

39. The method of claim 37, wherein the fluorinating agent is a compound having the formula: 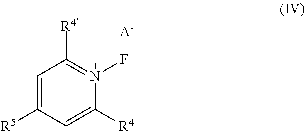
wherein
R⁴ and R⁴′ are each independently halogen, C₁₋₆ alkyl;
R⁵ is H or a C₁₋₆ alkyl; and
A⁻ is OTf⁻, BF₄⁻, or PF₆⁻.

40. The method of claim 39, wherein the fluorinating reagent is N-fluoro-2-4-6-trimethylpyridinium triflate.

41. The method of claim 28, wherein 1-5 equivalents of the fluorinating reagent is used.

42. The method of claim 28, wherein the mixture comprising a palladium (II) catalyst and a fluorinating reagent further comprises an additive and a solvent.

43. The method of claim 42, wherein the additive is N-Methyl-2-pyrrolidone (NMP).

44. The method of claim 42, wherein the solvent is trifluoromethyl benzene (PhCF₃), ethyl acetate (EtOAc) or CH₃CN.

45. The method of claim 42, wherein the solvent is CH₃CN.

46. The method of claim 28, wherein
Ar is a 2,3,5,6-tetrafluoro-4-(trifluoromethyl)phenyl group;
the palladium (II) catalyst is Pd(OTf)₂(CH₃CN)₄;
the fluorinating reagent is N-fluoro-2-4-6-trimethylpyridinium triflate;
the additive is NMP; and
the solvent is CH₃CN.

47. The method of claim 28, wherein reacting the compound having the formula (II) to form at least one of the ortho-fluorinated compounds having the formulae (IIa) and (IIb) is performed at a temperature under a heating condition.

48. The method of claim 47, the temperature is in the range of 8-120° C.

49. The method of claim 28, wherein the yield of monofluorinated product having the formula (IIa) is at least 50%, and the yield of difluorinated product having the formula (IIb) is less than 10%.

50. The method of claim 49, wherein the yield of monofluorinated product having the formula (IIa) is at least 80%, and the yield of difluorinated product having the formula (IIb) is less than 5%.

51. The method of claim 28, wherein the yield of difluorinated product having the formula (IIb) is at least 50%, and the yield of monofluorinated product having the formula (IIa) is less than 10%.

52. The method of claim 51, wherein the yield of difluorinated product having the formula (IIb) is at least 80%, and the yield of monofluorinated product having the formula (IIa) is less than 5%.

53. The method of claim 51, wherein the solvent is PhCF3.
